(19) United States
(12) Patent Application Publication
(10) Pub. No.: US 2016/0023126 A1
(43) Pub. Date: Jan. 28, 2016

(54) SPRAY DRYING TECHNIQUES

(71) Applicants: President and Fellows of Harvard College, Cambridge, MA (US); BASF SE, Ludwigshafen (DE)

(72) Inventors: Adam R. Abate, San Francisco, CA (US); Julian W.P. Thiele, Schwarzenbek (DE); David A. Weitz, Bolton, MA (US); Christian Holtze, Frankfurt (DE); Maike Windbergs, Saarbruecken (DE)

(73) Assignees: President and Fellows of Harvard College, Cambridge, MA (US); BASF SE, Ludwigshafen (DE)

(21) Appl. No.: 14/835,508

(22) Filed: Aug. 25, 2015

Related U.S. Application Data
(63) Continuation of application No. 13/330,793, filed on Dec. 20, 2011.
(60) Provisional application No. 61/485,026, filed on May 11, 2011.
(60) Provisional application No. 61/425,415, filed on Dec. 21, 2010.

(51) Int. Cl. B01D 1/18; A61K 31/58

(52) U.S. Cl. CPC B01D 1/18; A61K 31/58

(57) ABSTRACT

The present invention generally relates to microfluidics, and to spray drying and other drying techniques. In some aspects, an article containing one or more channels or microfluidic channels is used to mix one or more fluids prior to spray drying. The mixing may occur immediately before the fluids are expelled through a nozzle or other opening into a drying region of the spray dryer. In one set of embodiments, for example, a first fluid is exposed to a second fluid, then the fluids are exposed to air or other gases before being expelled through a nozzle. In certain instances, the first fluid may contain a dissolved species that may precipitate upon exposure to the second fluid; such precipitation may occur immediately before expulsion through a nozzle or other opening, thereby resulting in controlled precipitation as part of the spray drying process.

### RELATED APPLICATIONS

This application claims the benefit of U.S. Provisional Patent Application Ser. No. 61/425,415, filed Dec. 21, 2010, entitled “Spray Drying Techniques,” by

Abate, et al., and of U.S. Provisional Patent Application Ser. No. 61/485,026, filed May 11, 2011, entitled “Spray Drying Techniques,” by Abate, et al. Each of these is incorporated herein by reference.

### FIELD OF INVENTION

The present invention generally relates to microfluidics, and to spray drying and other drying techniques.

### BACKGROUND

Spray drying is a technique that is commonly used to dry substances, and is often used in diverse applications such as the spray drying of food (e.g., milk powder, coffee, tea, eggs, cereal, spices, flavorings, etc.), pharmaceutical compounds (e.g., antibiotics, medical ingredients, drugs, additives, etc.), industrial compounds (e.g., paint pigments, ceramic materials, catalysts, etc.), or the like. In spray drying, a substance to be dried is typically expelled from a nozzle into a region that is dried and/or heated in order to cause the drying of the substance to occur. The substance is often liquid, although other substances may also be dried, for example wet or slushy solid materials. The region used for drying may contain air or nitrogen, and in some cases is heated. The substance is typically broken up into smaller pieces, e.g., using a nozzle, to increase the exposed surface area and thus decrease the drying time of the substance. However, such drying techniques may be hard to control, e.g., when a consistent size distribution of dried product is desired.

### SUMMARY OF THE INVENTION

The present invention generally relates to microfluidics, and to spray drying and other drying techniques. The subject matter of the present invention involves, in some cases, interrelated products, alternative solutions to a particular problem, and/or a plurality of different uses of one or more systems and/or articles.

In one aspect, the present invention is generally directed to a spray dryer. In one set of embodiments, the spray dryer includes an article comprising a first microfluidic channel having an opening as a nozzle, and a second microfluidic channel intersecting the first microfluidic channel at an intersection upstream of the nozzle. In some cases, the spray dryer may also include a drying region that receives output from the nozzle.

In accordance with another set of embodiments, the spray dryer may include an article comprising one more microfluidic channels that together have an average cross-sectional dimension of less than about 1 mm and a total length of at least about 10 mm. The spray dryer can also include a drying region that receives output from the nozzle. In some cases, at least one of the microfluidic channels has an opening in the article acting as a nozzle.

The spray dryer, in yet another set of embodiments, includes an article comprising a fluidic channel having a cross-sectional aspect ratio of at least about 3:1, and a drying region that receives output from the nozzle. The spray dryer may also contain a fluidic channel having an opening that acts as a nozzle.

In still another set of embodiments, the spray dryer includes a fluidic channel containing a first liquid and a second liquid, an outlet of the fluidic channel acting as a nozzle, and a drying region that receives output from the nozzle. In some embodiments, proximate to the outlet, the second liquid surrounds the first liquid such that the first liquid does not contact a wall of the fluidic channel.

In another set of embodiments, the spray dryer includes an article comprising a first fluidic channel having an opening acting as a nozzle, a second fluidic channel intersecting the first fluidic channel at an intersection upstream of the nozzle, and a third fluidic channel intersecting the first fluidic channel at the intersection upstream of the nozzle. The spray dryer may also include a drying region that receives output from the nozzle.

The spray dryer, in accordance with yet another set of embodiments, includes an article comprising an elastomeric polymer, the article comprising a fluidic channel having an opening acting as a nozzle, and a drying region that receives output from the nozzle.

In still another set of embodiments, the spray dryer includes a mechanically deformable article comprising a fluidic channel having an opening acting as a nozzle, and a drying region that receives output from the nozzle.

In accordance with still another set of embodiments, the spray dryer may include at least 10 articles each comprising a fluidic channel having an opening acting as a nozzle, and a drying region that receives output from the nozzles of the at least 10 articles. The spray dryer, in yet another set of embodiments, includes an article comprising at least 10 fluidic channels, each having an opening acting as a nozzle, and a drying region that receives output from each of the at least 10 nozzles. According to still another set of embodiments, the spray dryer includes at least 10 articles, each article comprising, an article comprising a fluidic channel having an opening acting as a nozzle, and a drying region that receives output from the nozzle.

The spray dryer, in another set of embodiments, includes a quasi-2-dimensional article comprising a fluidic channel having an opening acting as a nozzle, and a drying region that receives output from the nozzle.

In another aspect, the present invention is generally directed to a method of spray drying, e.g., a fluid or a liquid. The method, in accordance with one set of embodiments, includes acts of providing a first liquid comprising a species dissolved in the first liquid, within a fluidic channel, exposing the first liquid to a second liquid for a period of time of no more than about 30 seconds, and spraying the first liquid and the second liquid into a drying region external of the fluidic channel. In some embodiments, the species is not substantially soluble in the second liquid.

In another set of embodiments, the method include acts of, within a microfluidic channel, exposing a first liquid to a second liquid, and expelling the first liquid and the second liquid into a drying region external of the microfluidic channel.

The method, in yet another set of embodiments, includes acts of providing a channel containing a liquid delineated by a first bolus upstream of the liquid and delineated by a second bolus downstream of the liquid, and expelling the liquid into a drying region external of the fluidic channel.

In another set of embodiments, the method includes acts of providing a first liquid comprising a species dissolved in the first liquid; within a fluidic channel, exposing the first liquid to a second liquid that cannot substantially dissolve the species for a period of time of no more than 30 seconds; and spraying the first liquid and the second liquid into a drying region external of the fluidic channel. The method, in accordance with yet another set of embodiments, includes acts of providing a first liquid comprising a species dissolved in the first liquid; within a fluidic channel, causing the species to precipitate from the first liquid; and spraying the first liquid and the species into a drying region external of the fluidic channel.

The method, in still another set of embodiments, includes acts of providing a double emulsion within a microfluidic channel; and spraying the double emulsion into a drying region external of the microfluidic channel.

In yet another set of embodiments, the method includes acts of passing a liquid through a microfluidic channel wherein the liquid flow through the channel has a Reynolds number of at least about 1; and expelling the liquid into a drying region external of the fluidic channel.

The method, in accordance with another set of embodiments, includes exposing a first liquid to a second liquid, expelling the first liquid and the second liquid into a drying region external of the microfluidic channel to produce a product, and collecting the product in a collection chamber having a volume of less than about 20 ml.

In another aspect, the present invention encompasses methods of making one or more of the embodiments described herein, for example, spray drying and other drying techniques involving microfluidics. In still another aspect, the present invention encompasses methods of using one or more of the embodiments described herein, for example, spray drying and other drying techniques involving microfluidics.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrates various microfluidic devices in accordance with certain embodiments of the invention;

FIGS. 2A-2C illustrate a microfluidic device having a pressurized fluid, in another embodiment of the invention;

FIGS. 3A-3D illustrate spray profiles of certain embodiments of the invention;

FIGS. 4A-4E illustrate the effect of solvent, in accordance with still other embodiments of the invention;

FIGS. 5A-5E illustrate the effect of spatial sampling, in yet another embodiment of the invention;

FIGS. 6A-6F illustrate inhibition of crystallization in still other embodiments of the invention;

FIGS. 7A-7D illustrate comparable results using a conventional spray dryer, in accordance with certain embodiments of the invention;

FIG. 8 is a schematic diagram of a spray dryer in accordance with still another embodiment of the invention; and

FIGS. 9A-9C illustrate another embodiment of the invention having reduced or no fouling of a precipitant.

### DETAILED DESCRIPTION

The present invention generally relates to microfluidics, and to spray drying and other drying techniques. In some aspects, an article containing one or more channels or microfluidic channels is used to mix one or more fluids prior to spray drying. The mixing may occur immediately before the fluids are expelled through a nozzle or other opening into a drying region of the spray dryer. In one set of embodiments, for example, a first fluid is exposed to a second fluid, then the fluids are exposed to air or other gases before being expelled through a nozzle. In certain instances, the first fluid may contain a dissolved species that may precipitate upon exposure to the second fluid; such precipitation may occur immediately before expulsion through a nozzle or other opening, thereby resulting in controlled precipitation as part of the spray drying process.

Thus, in certain aspects, the present invention is generally directed to a spray dryer. In a spray dryer, a fluid (typically a liquid) is expelled into a drying region in order to at least partially dry the fluid. In some cases, particles or solids are formed as a result of drying of the fluid. The drying region may contain a gas that is heated and/or has reduced humidity in order to facilitate drying. Examples of gases that may be used for drying include, but are not limited to, air or nitrogen. The fluid may be expelled into the drying region, according to some embodiments, via a nozzle or other opening, which can be used to cause the fluid to form droplets. The droplets increase the amount of surface area of contact between the fluid and the surrounding gas, thereby increasing the rate of drying. In some cases, the fluid may be combined with other fluids when directed through the nozzle; for example, the fluid may be combined with air or other gases in order to cause the fluid to form droplets. Spray drying is used in a variety of applications where drying is desired. For example, spray drying may be used to dry thermally sensitive materials or thermally degradable materials, and/or to dry a fluid at a controlled rate. In some cases, spray drying may also be used to create relatively uniform particles, e.g., due to drying of the fluid at a controlled rate. Other non-limiting examples of materials and applications suitable for spray drying include those described above, and herein.

In some embodiments, the present invention is directed to spray dryers in which one or more articles containing various channels are used to prepare a fluid prior to, or as part of, the spray drying process. Some or all of the channels can be microfluidic channels. The channels may be used to expel a fluid into a drying region in a spray dryer, and in some cases, additional channels may be connected to the channel, for example, to add or mix fluids, cause precipitation of a species within a fluid, manipulate a droplet or other species, or the like. Various non-limiting examples of these are discussed below.

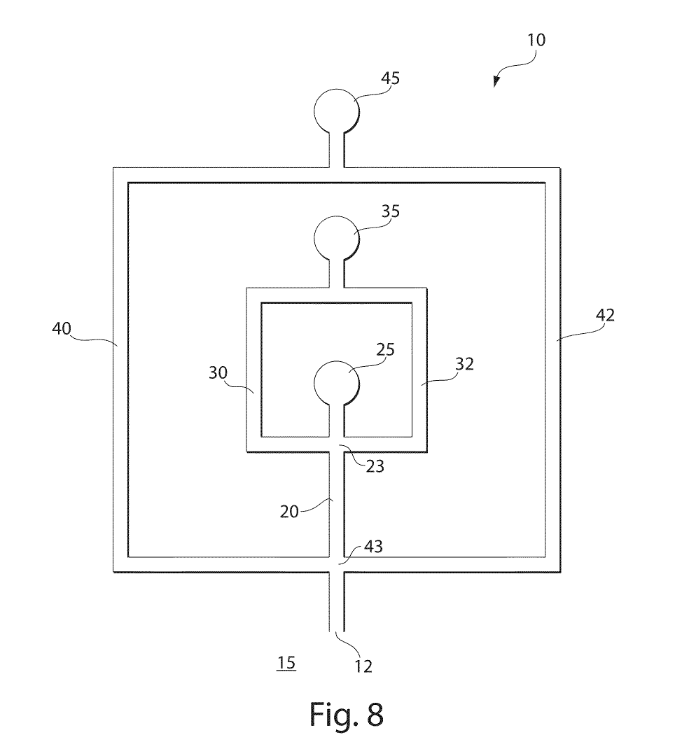

A non-limiting example of such a spray dryer is now discussed with reference to FIG. 8. In this figure, fluidic system 10 is illustrated. In some embodiments, some or all of the channels shown in fluidic system 10 may be microfluidic channels, e.g., as is discussed herein. Fluidic system 10, in this example, includes a nozzle 12 which is positioned at the end of first channel 20, although in other embodiments, other openings can be used as well, for example, an opening in the side of a channel. In this illustration, first channel 20 is generally straight, although in other embodiments, first channel 20 can have a variety of other geometries, e.g., any number of bends, zigzags, valves, or other channel elements may be present as part of first channel 20. Fluid entering first channel 20 enters from first fluid source 25. First fluid source 25 may be connected to a reservoir, a pump, a syringe, a pipette, or another source of a suitable fluid. The fluid can then pass through first channel 20 (or a portion of the channel, in some cases), before being expelled through nozzle 12. The fluid then passes into drying region 15, where the fluid can then be dried, e.g., by exposure to a gas that is heated and/or has reduced humidity.

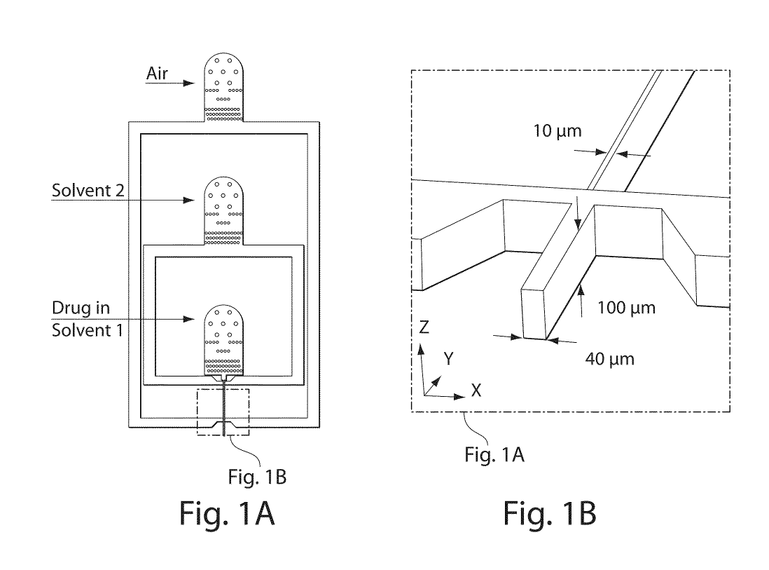

Also shown in FIG. 1 are second channel 30 and third channel 32, each connecting to second fluid source 35. The second fluid arising from second fluid source 35 can be the same or different than the first fluid arising from first fluid source 25. In this example, second channel 30 and third channel 32 each intersect first channel 20 at the same junction 23, each meeting at right angles to first channel 20, such that second channel 30 and third channel 32 come into contact with first channel 20 oppositely of each other. In this example, second channel 30 and third channel 32 are each suitably routed from second fluid source 35 around first fluid source 25 in order to reach fluid channel 20 at junction 23. Although two channels are shown here, this is by way of example only, and in other embodiments, there may be other numbers of channels between second fluid source 35 and first channel 20, e.g., 1 channel, 3 channels, 4 channels, 5 channels, 10 channels, etc. In addition, in some cases, if more than one channel is present, the channels need not all interest first channel 20 at a common intersection.

Such a configuration may be used, for example, to cause a first fluid from first fluid source 25 and a second fluid from second fluid source 35 to come into contact with each other at or downstream of junction 23. The first fluid and the second fluid may be miscible or immiscible. For example, if the first fluid and the second fluid are immiscible, the first fluid may be caused to form droplets within the second fluid. Droplet formation can be controlled, for example, by controlling the properties of the fluids, the flow rates of the fluids entering junction 23, or the like. As another example, the first fluid and the second fluid may be at least partially miscible with each other. For instance, the first fluid can contain a species dissolved therein, where the species is not soluble (or is not soluble to the same degree) in the second fluid. For example, the species may be one that is not substantially soluble in the second liquid; as a specific example, the species may have a solubility that is at least 1, 2, 3, 4, or 5 orders of magnitude (powers of 10) lower than the solubility of the species in the first liquid. Exposure of the species to the second fluid starting at junction 23 may then cause at least some of the species to begin precipitating within first channel 20, for example, to be carried downstream as a solid precipitant towards nozzle 12.

Additionally, shown in FIG. 1 are third fluid source 45, connecting to fourth channel 40 and fifth channel 42. Fourth channel 40 and fifth channel 42 each intersect first channel 20 at junction 43, which may be upstream or downstream of junction 23, depending on the embodiment. At junction 43, fourth channel 40 and fifth channel 42 intersect first channel 20 at right angles, and oppositely of each other. Fluid source 45 may be used to deliver a third fluid, for example, which is delivered to first channel 20 just before the fluid within first channel 20 exits through nozzle 12. The third fluid may be used, as a specific non-limiting example, to cause the other fluids within first channel 20 to break up to form individual or discrete droplets, which are then expelled out of first channel 20 as discrete droplets into drying region 15 for drying purposes.

Fourth channel 40 and fifth channel 42 may be suitable routed from third fluid source 45 around the other elements of fluidic system 10 to reach junction 43. Although this example only illustrates two channels, this is for illustrative purposes only; in other embodiments, there may be other numbers of channels between third fluid source 45 and first channel 20, e.g., 1 channel, 3 channels, 4 channels, 5 channels, 10 channels, etc. In addition, in some cases, if more than one channel is present, the channels need not all interest first channel 20 at a common intersection.

Accordingly, various aspects of the present invention are directed to spray dryers where fluids are prepared using one or more channels such as microfluidic channels, before being expelled into a suitable drying region. The drying region may be open, e.g., open to the atmosphere, or closed, for example, partially or completely surrounded by a drying chamber into which the fluids are expelled. For example, a drying chamber can be formed of glass, plastic, or any other suitable material which can be used to at least partially contain or enclose a suitable drying gas for drying fluids expelled into the drying region. The drying gas may be air, nitrogen, carbon dioxide, argon, or other suitable gases. In some embodiments, the gas is chosen so as to be relatively inert or unreactive to the expelled fluids; however, in other embodiments, the gas may react with one or more of the expelled fluids. The drying gas can also be dehumidified using various techniques, for example, refrigeration or condensing cycles, electronic methods (e.g., Peltier heat pumps), desiccants (e.g., phosphorus pentoxide), or hygroscopic materials. In some embodiments, the relative humidity within the drying region is no more than about 50%, no more than about 40%, no more than about 35%, no more than about 30%, no more than about 25%, no more than about 20%, no more than about 15%, no more than about 10%, or no more than about 5%. Other techniques for controlling the relative humidity of a region will be known to those of ordinary skill in the art.

In some cases, the drying region is heated, e.g., using one or more heaters. The temperature of the drying region may be chosen, for example, to allow partial or complete drying of the expelled fluids to occur (depending on the application), in some cases without causing adverse degradation or reaction with the expelled fluids. For example, the heater may be used to heat the drying region to a temperature of at least about 30° C., at least about 40° C., at least about 60° C., at least about 80° C., at least about 100° C., at least about 125° C., at least about 150° C., at least about 200° C., at least about 300° C., at least about 400° C., at least about 500° C., etc. Any suitable method may be used to heat the drying region. For example, the drying region may be heated using induction heating, burning of a fuel, exposure to radiation (e.g., infrared radiation), chemical reaction, or the like.

The spray dryer also may contain an article containing one or more channels such as microfluidic channels. The article can be formed, for example, from polymeric, flexible, and/or elastomeric polymers and/or other materials, e.g., silicone polymers such as polydimethylsiloxane (“PDMS”). In some embodiments, the article may comprise or even consist essentially of such polymers and/or other materials. Other examples of potentially suitable polymers and other materials are discussed in detail below. The article may be planar, or non-planar in some embodiments (e.g., curved). The article can be formed from a material that is at least partially mechanically deformable in some cases, e.g., such that the article can be visibly mechanically deformed by an average person without the use of tools. In other embodiments, however, the article may be formed of more relatively rigid materials such that the article is not as mechanically deformable.

There may be one or more openings in one or more of the channels that are used to expel fluids contained therein into the drying region, or into more than one drying region in some cases. The openings can be, for instance, a simple opening or a hole in the side of a channel, an open end of a channel, or there may be an additional structure associated with the opening that the fluids pass through before being expelled into a drying region, for example, a pipe or a tube having varying cross sectional area that can be used to direct or modify the flow of the fluid. The opening can act as a nozzle through which a fluid can be expelled from the channel into the drying region. In some cases, the opening is constructed such that fluid passing therethrough forms individual or discrete droplets. For example, in certain embodiments, the opening may be constructed and arranged to cause a fluid to form a spray or a mist of droplets. In other embodiments, the droplets can be expelled as a regular or steady stream of droplets, e.g., a single file stream of droplets.

There can be any number of channels, including microfluidic channels, within the article, and the channels may be arranged in any suitable configuration. The channels may be all interconnected, or there can be more than one network of channels present. In addition, there may be one or more openings in one or more of the channels that are used to expel fluids contained therein, as discussed above. In some cases, there are a relatively large number and/or a relatively large length of channels present in the article. For example, in some embodiments, the channels within an article, when added together, can have a total length of at least about 100 micrometers, at least about 300 micrometers, at least about 500 micrometers, at least about 1 mm, at least about 3 mm, at least about 5 mm, at least about 10 mm, at least about 30 mm, at least 50 mm, at least about 100 mm, at least about 300 mm, at least about 500 mm, at least about 1 m, at least about 2 m, or at least about 3 m in some cases. As another example, an article can have at least 1 channel, at least 3 channels, at least 5 channels, at least 10 channels, at least 20 channels, at least 30 channels, at least 40 channels, at least 50 channels, at least 70 channels, at least 100 channels, etc.

In some embodiments, at least some of the channels within the article are microfluidic channels. “Microfluidic,” as used herein, refers to a device, article, or system including at least one fluid channel having a cross-sectional dimension of less than about 1 mm. The “cross-sectional dimension” of the channel is measured perpendicular to the direction of net fluid flow within the channel. Thus, for example, some or all of the fluid channels in an article can have a maximum cross-sectional dimension less than about 2 mm, and in certain cases, less than about 1 mm. In one set of embodiments, all fluid channels in an article are microfluidic and/or have a largest cross sectional dimension of no more than about 2 mm or about 1 mm. In certain embodiments, the fluid channels may be formed in part by a single component (e.g. an etched substrate or molded unit). Of course, larger channels, tubes, chambers, reservoirs, etc. can be used to store fluids and/or deliver fluids to various elements or systems in other embodiments of the invention. In one set of embodiments, the maximum cross-sectional dimension of the channels in an article is less than 500 microns, less than 200 microns, less than 100 microns, less than 50 microns, or less than 25 microns.

A “channel,” as used herein, means a feature on or in an article or substrate that at least partially directs flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlets and/or outlets or openings. A channel may also have an aspect ratio (length to average cross sectional dimension) of at least 2:1, more typically at least 3:1, 4:1, 5:1, 6:1, 8:1, 10:1, 15:1, 20:1, or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to net fluid flow of less than about 5 mm or 2 mm, or less than about 1 mm, less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases, the dimensions of the channel are chosen such that fluid is able to freely flow through the article or substrate. The dimensions of the channel may also be chosen, for example, to allow a certain volumetric or linear flow rate of fluid in the channel. Of course, the number of channels and the shape of the channels can be varied by any method known to those of ordinary skill in the art. In some cases, more than one channel may be used. For example, two or more channels may be used, where they are positioned adjacent or proximate to each other, positioned to intersect with each other, etc.

In one set of embodiments, the channels within the article are arranged in a quasi-2-dimensional pattern. In a “quasi-2-dimensional pattern,” the channels within the article are constructed and arranged such that at least one plane can be defined relative to the article such that, when all of the channels within the article are “shadowed” or perpendicularly projected onto the plane, any two channels that appear to be fluidically connected are, in fact, fluidically connected (i.e., there are no “bridges” within the article separating those fluids in separate channels). Such articles are useful in certain cases, for example, due to their ease of manufacturing, creation, or preparation.

In certain embodiments, one or more of the channels within the article may have an average cross-sectional dimension of less than about 10 cm. In certain instances, the average cross-sectional dimension of the channel is less than about 5 cm, less than about 3 cm, less than about 1 cm, less than about 5 mm, less than about 3 mm, less than about 1 mm, less than 500 micrometers, less than 200 micrometers, less than 100 micrometers, less than 50 micrometers, or less than 25 micrometers. The “average cross-sectional dimension” is measured in a plane perpendicular to net fluid flow within the channel. If the channel is non-circular, the average cross-sectional dimension may be taken as the diameter of a circle having the same area as the cross-sectional area of the channel. Thus, the channel may have any suitable cross-sectional shape, for example, circular, oval, triangular, irregular, square, rectangular, quadrilateral, or the like. In some embodiments, the channels are sized so as to allow laminar flow of one or more fluids contained within the channel to occur.

The channel may also have any suitable cross-sectional aspect ratio. The “cross-sectional aspect ratio” is, for the cross-sectional shape of a channel, the largest possible ratio (large to small) of two measurements made orthogonal to each other on the cross-sectional shape. For example, the channel may have a cross-sectional aspect ratio of less than about 2:1, less than about 1.5:1 , or in some cases about 1:1 (e.g., for a circular or a square cross-sectional shape). In other embodiments, the cross-sectional aspect ratio may be relatively large. For example, the cross-sectional aspect ratio may be at least about 2:1, at least about 3:1, at least about 4:1, at least about 5:1, at least about 6:1, at least about 7:1, at least about 8:1, at least about 10:1, at least about 12:1, at least about 15:1, or at least about 20:1. Relatively large cross-sectional aspect ratios are useful in accordance with some embodiments, as is discussed herein, for preventing or minimizing contact between a fluid within a channel and one or more walls within the channel.

As mentioned, the channels can be arranged in any suitable configuration within the article. Different channel arrangements may be used, for example, to manipulate fluids, droplets, and/or other species within the channels. For example, channels within the article can be arranged to create droplets (e.g., discrete droplets, single emulsions, double emulsions or other multiple emulsions, etc.), to mix fluids and/or droplets or other species contained therein, to screen or sort fluids and/or droplets or other species contained therein, to split or divide fluids and/or droplets, to cause a reaction to occur (e.g., between two fluids, between a species carried by a first fluid and a second fluid, or between two species carried by two fluids to occur), or the like. As a specific example, two or more channels can be arranged to cause “flow-focusing” of different fluids within the channels to form droplets.

Non-limiting examples of systems for manipulating fluids, droplets, and/or other species are discussed below. Additional examples of suitable manipulation systems can also be seen in U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., now U.S. Pat. No. 7,708,949, issued May 4, 2010; U.S. patent application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009; and U.S. patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007; each of which is incorporated herein by reference in its entirety.

Fluids may be delivered into channels within an article via one or more fluid sources. Any suitable source of fluid can be used, and in some cases, more than one source of fluid is used. For example, a pump, gravity, capillary action, surface tension, electroosmosis, centrifugal forces, etc. may be used to deliver a fluid from a fluid source into one or more channels in the article. Non-limiting examples of pumps include syringe pumps, peristaltic pumps, pressurized fluid sources, or the like. The article can have any number of fluid sources associated with it, for example, 1, 2, 3, 4, 5, 6, 7, 8, 9, 10, etc., or more fluid sources. The fluid sources need not be used to deliver fluid into the same channel, e.g., a first fluid source can deliver a first fluid to a first channel while a second fluid source can deliver a second fluid to a second channel, etc.

In some cases, two or more channels are arranged to intersect at one or more junctions. There may be any number of fluidic channel junctions within the article, for example, 2, 3, 4, 5, 6, etc., or more junctions. As a specific non-limiting example, in some embodiments, the article includes a first channel having an opening as a nozzle, and a second channel intersecting the first channel. The junction of the first channel and the second channel may be upstream of the nozzle, e.g., a fluid within the first channel may pass by or through the junction before being expelled from the nozzle into the drying region. Such a configuration can be useful, for example, to mix a first fluid in the first channel with a second fluid in the second channel, to cause a reaction between a species contained within the first fluid within the first channel with the second fluid and/or a second species contained within the second fluid in the second channel, to cause discrete droplets of the first fluid to form within the second fluid, to cause the formation of a double or other multiple emulsion between the first fluid and the second fluid, or the like.

In one set of embodiments, there may be one, two, three, or more channels arranged in a “flow focusing” configuration in the article, e.g., in which a first fluid in a first channel is sheathed or surrounded by a second fluid delivered using additional channels (e.g., a second channel and sometimes a third channel or additional channels) in order to cause the first fluid to form discrete droplets contained within the second fluid. The first fluid and the second fluid can be miscible or immiscible. Channel configurations to create such discrete droplets may be found, for example, in U.S. patent application Ser. No. 11/024,228, filed Dec. 28, 2004, entitled “Method and Apparatus for Fluid Dispersion,” by Stone, et al., now U.S. Pat. No. 7,708,949, issued May 4, 2010, incorporated herein by reference in its entirety. As a non-limiting example, there may be a first channel having an opening as a nozzle, and second and third channels each intersecting the first channel at a common junction. (In other embodiments of the invention, there may be more or fewer additional channels present.) Fluid within the second and third channels can arise from a common fluid source or from two different fluid sources, and the fluids within the second and third channels can be the same or different. One or both the second channel and the third channel may each meet the first channel at a substantially right angle, or at another suitable angle. In some cases, the second channel and the third channel may meet the first channel substantially opposite of each other, although in other cases, the channels may not all intersect at the same junction.

As another example, in one set of embodiments, a double emulsion or other multiple emulsion may be formed in the channel, e.g., using configurations such as those described in U.S. patent application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009, or U.S. patent application Ser. No. 12/058,628, filed Mar. 28, 2008, entitled “Emulsions and Techniques for Formation,” by Chu, et al., now U.S. Pat. No. 7,776,927, issued Aug. 17, 2010, each incorporated herein by reference in its entirety. Other suitable techniques for preparing double emulsions are disclosed in International Patent Application No. PCT/US2010/000763, filed Mar. 12, 2010, entitled “Controlled Creation of Multiple Emulsions,” by Weitz, et al., published as WO 2010/104604 on Sep. 16, 2010; or International Patent Application No. PCT/US2010/047458, filed Sep. 1, 2010, entitled “Multiple Emulsions Created Using Junctions,” by Weitz, et al., each incorporated herein by reference in its entirety.

In certain embodiments, the channels are arranged such that a first fluid in a first channel, upon the addition of other fluids within the channel, does not come into contact with a wall of the channel. For instance, after introduction of the second fluid to the channel containing the first fluid, the first fluid may not come into contact with any of the walls defining the channel and is thus completely surrounded or sheathed by the second fluid when viewed in cross-section; e.g., there is at least one other fluid between the first fluid and the walls of the channel. The separation of the first fluid from the walls of the channel may be due to the introduction of the second fluid, due to the degree of miscibility of the first fluid and the second fluid, due to the shape or geometry of the channel containing the fluids, etc. In some embodiments, the first fluid may also contain one or more species therein, which also cannot come into contact with any of the walls defining the channel.

The first fluid can be present within the second fluid as a continuous stream, or as discrete droplets. The first fluid may be prevented from coming into contact with a wall of the channel at least proximate an opening within the channel, e.g., that is used to expel fluids contained therein into a drying region. In some embodiments, the first fluid is prevented from coming into contact with a wall of the channel substantially throughout the length of the channel. By preventing the first fluid from contacting the walls of the channel, reactions or interactions between the first fluid and the walls of the channel may be reduced or eliminated. For instance, the first fluid may contain a species (e.g., dissolved or suspended therein) that is able to bind to (or “foul”) a wall of the channel if the species comes into contact with the wall; by preventing, reducing, or minimizing contact between the first fluid and the wall, the ability of the species to bind to the wall is reduced or eliminated. Such binding may be specific or non-specific.

In certain instances, the channel may be shaped to assist in preventing the first fluid from contacting any walls defining the fluidic channel. For example, in some cases, the channel may be one that has a relatively large cross-sectional aspect ratio, e.g., as in an oval or a rectangle. As discussed herein, as examples, the cross-sectional aspect ratio of the channel may be at least about 2:1, at least about 3:1, at least about 4:1, at least about 5:1, at least about 6:1, at least about 7:1, at least about 8:1, at least about 10:1, at least about 15:1, at least about 20:1, etc. Such channels may be useful, for example, since some of the walls of the channel are positioned relatively far away from the first fluid within the channel.

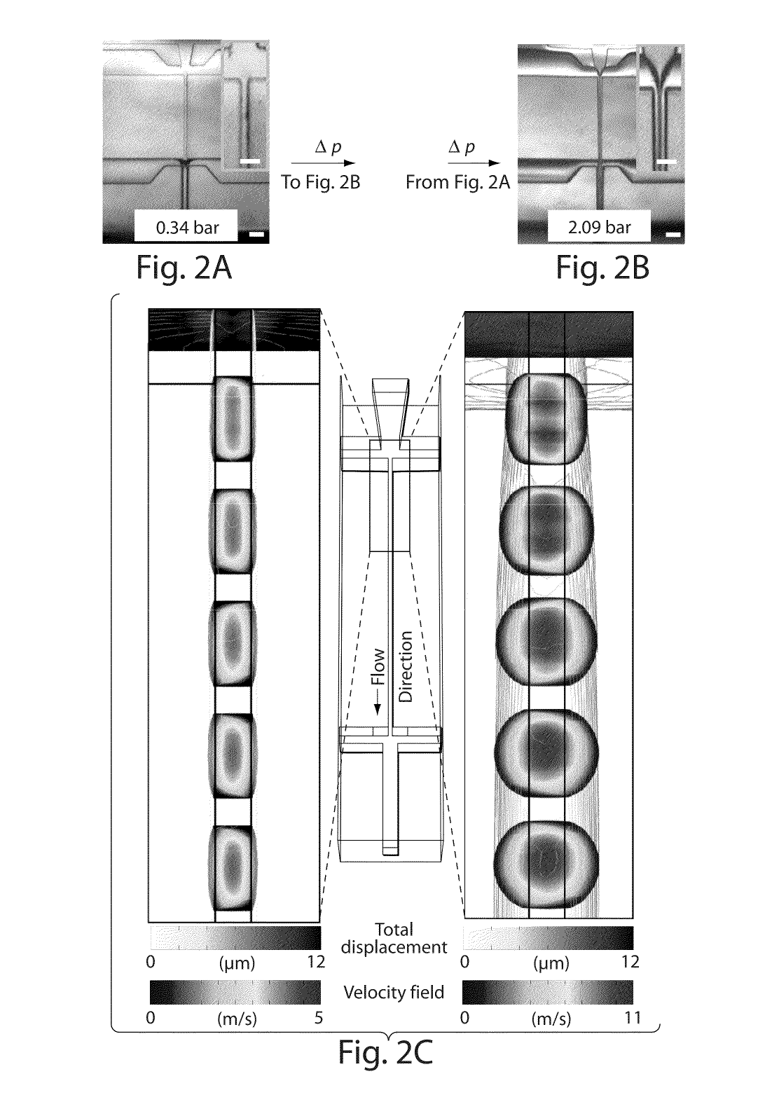

In addition, in some embodiments, as discussed herein, the channels may also be expanded upon introduction of a fluid, e.g., a pressurized fluid, which may also help to prevent the first fluid from contacting any walls defining the fluidic channel. For example, the channels may become distended due to fluid therein, which causes at least some of the channel walls to bow away from any fluids contained therein. See, e.g., FIG. 2 and the examples below.

The channel may also be coated in some embodiments. For example, the coating may render the walls (or a portion thereof) of the channel more hydrophobic or more hydrophilic, depending on the application. As a specific non-limiting example, the first fluid may be relatively hydrophilic and the channel walls may be relatively hydrophobic, and/or coated to render the walls more hydrophobic, such that the first fluid is generally repelled (does not wet) the walls of the channel, thereby assisting in preventing the first fluid from contacting the hydrophobic walls defining the fluidic channel. As another example, the first fluid may be relatively hydrophobic and the channel walls may be relatively hydrophilic. Typically, a “hydrophilic” material or surface is one that wets water, e.g., water on such a surface has a contact angle of less than 90°, while a “hydrophobic” material or surface has a contact angle of greater than 90°. However, hydrophobicity may also be determined in other embodiments in a relative sense, i.e., a first material may be more hydrophilic than a second material (e.g., have a smaller contact angle), although the materials may both be hydrophilic or both be hydrophobic.

Any suitable method may be used to coat or treat the walls (or a portion thereof) of a channel. For instance, a wall can be treated with oxygen plasma treatment, or coated with a sol-gel material that can be used to alter the hydrophobicity of the wall. A portion of the sol-gel may be exposed to light, such as ultraviolet light, which can be used to induce a chemical reaction in the sol-gel that alters its hydrophobicity. The sol-gel can include a photoinitiator which, upon exposure to light, produces radicals. Optionally, the photoinitiator is conjugated to a silane or other material within the sol-gel. The radicals so produced may be used to cause a condensation or polymerization reaction to occur on the surface of the sol-gel, thus altering the hydrophobicity of the surface. As another non-limiting example, a metal oxide may be coated onto a wall to alter its hydrophobicity. Still other examples are disclosed below, and in International Patent Application No. PCT/US2009/000850, filed Feb. 11, 2009, entitled “Surfaces, Including Microfluidic Channels, With Controlled Wetting Properties,” by Abate, et al., published as WO 2009/120254 on Oct. 1, 2009, and U.S. patent application Ser. No. 12/733,086, filed Feb. 5, 2010, entitled “Metal Oxide Coating on Surfaces,” by Weitz, et al., published as U.S. Patent Application Publication No. 2010/0239824 on Sep. 23, 2010, each of which is incorporated herein by reference in its entirety.

As still another example, an additional liquid may be added to prevent or remove a precipitant in contact with the walls of a channel. For example, in one set of embodiments, a first fluid containing a species such as a drug is exposed to a second species that causes the species to precipitate. Some of the precipitant may contact one or more walls of the channel, thereby fouling the walls and the channel. However, a third fluid can also be added, e.g., from another inlet, in order to remove the fouling from the walls of the channels. In some cases, the third fluid may be used to sheath the other fluids and thereby prevent the other fluids from contacting the walls of the channels. In some cases, the third fluid may also be selected to be one in which the precipitant is able to dissolve, thereby reducing or eliminating the potential of the precipitant to stay solid and/or precipitated on the walls of the channels.

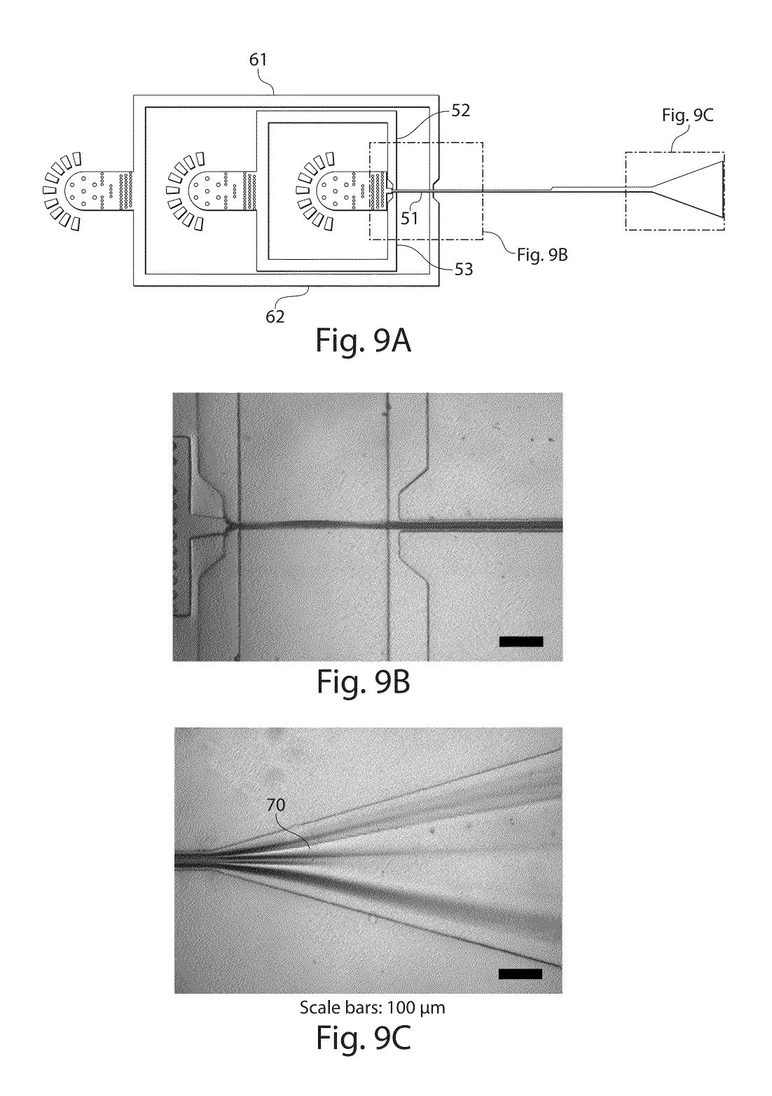

One non-limiting example can be seen in FIG. 9. FIG. 9A illustrates a schematic diagram of an article containing microfluidic channels in accordance with one embodiment of the invention; FIGS. 9B and 9C are photomicrographs taken of the left and right boxed sections in FIG. 9A. The scale bars represent 100 micrometers in these figures. In these experiments, a drug solution (e.g., saturated danazol in isopropyl alcohol) was added to the center channel 51 with water in the side channels 52, 53. The two phases form a jet which extends into the second junction where additional isopropyl alcohol is added from side channels 61, 62. In some cases, the fluidic stream may be broken up to from droplets, e.g., upon addition of air or another suitable gas (not shown in this figure). In FIG. 9C, an expanded view of the resulting fluidic stream is illustrated. In this figure, the darker shadows 70 are caused by the precipitation of the danazol at the interface of the isopropyl alcohol and water streams due to diffusion-based mixing. However, due to the sheath of the isopropyl alcohol surrounding the two inner flows, the precipitating danazol does not come into contact with the walls of the channels. In certain embodiments, there may be one, two, three, or more channels arranged to deliver a gas to a liquid contained within a channel. The gas can be, for example, air, oxygen, nitrogen, carbon dioxide, argon, and/or another gas. In some cases, the gas is also dehumidified. The gas may arise from one or more suitable gas sources, e.g., as discussed herein. In some cases, a first channel can contain a liquid (or more than one liquid), and a gas delivered to the first channel using additional channels (e.g., a second channel and a third channel). One or both the second channel and the third channel may each meet the first channel at a substantially right angle (or at another angle), and in some embodiments, the second channel and the third channel meet the first channel substantially opposite of each other. The gas can be used, for example, to cause the liquid to form discrete droplets, e.g., to be expelled from a nozzle or an opening in the channel to a drying region. In certain embodiments, an article may contain more than one such channel arrangement. An example of this is illustrated in FIG. 1 with first channel 20 and first fluid source 25; second channel 30 and third channel 32 may introduce a second fluid from second fluid source 35 to first channel 20, while fourth channel 40 and fifth channel 42 may introduce a gas (or another third fluid) from third fluid source 45 to first channel 20.

As another non-limiting example, a first channel having an opening as a nozzle may be intersected by second and third channels at a common junction (in other embodiments of the invention, there may be more or fewer additional channels present). The common junction may be at or proximate the opening in certain cases. In some embodiments, the gas is heated (e.g., to speed drying), although in other embodiments, the gas may be at ambient temperature or even cooled in some cases. Any suitable gas can be used, for example, air, oxygen, nitrogen, carbon dioxide, argon, etc., or any combination of these and/or other gases. The gas may be chosen to be reactive or inert to the liquid(s) or species contained therein, depending on the application.

The gas can be delivered to the first channel, for example, via the second and/or third channels. The gas can be at ambient pressure, or the gas may be pressurized in some instances. For instance, the pressure of the incoming gas may be at least about 0.01 bar, at least about 0.03 bar, at least about 0.05 bar, at least about 0.07 bar, at least about 0.1 bar, at least about 0.2 bar, at least about 0.3 bar, at least about 0.4 bar, at least about 0.5 bar, at least about 0.7 bar, at least about 1 bar, at least about 2 bar, at least about 3 bar, at least about 4 bar, or at least about 5 bar. The introduction of gas to the liquid can cause the liquid to break up into discrete droplets, and in some cases, such that a spray or a mist of droplets is formed.

In some embodiments, the droplets have an average diameter of less than about 1 cm, less than about 7 mm, less than about 5 mm, less than about 3 mm, than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 7 micrometers, less than about 5 micrometers, less than about 3 micrometers, or less than about 1 micrometer. As discussed above, in certain instances, smaller droplets may be preferable due to the larger surface area to volume ratio such droplets have, relative to larger droplets, which may increase drying speed, uniformity of drying, or other drying characteristics within the spray dryer.

In another set of embodiments, electrospray techniques are used. For example, a fluid can be broken up to form droplets using an electric field or other suitable electrospray techniques. Such techniques may be used instead of, or in combination with, the use of air or other gases to cause the formation of droplets as discussed above. For instance, in some cases, a relatively high electric field or voltage can be applied to a liquid. In some embodiments, the liquid is induced to form a Taylor cone (which may decreases in cross-sectional dimension upon exiting a channel, e.g., in a downstream direction), which emits a liquid jet through its apex, causing highly charged liquid droplets to break off the Taylor cone as a series of droplets. In some instances, the droplets can become radially dispersed due to Columbic repulsion. A Taylor cone is a shape that a fluidic stream of an at least partially electrically conductive fluid assumes when exposed to an externally applied inductive electric field, as is known to those of ordinary skill in the art. In the formation of a Taylor cone, an electric field can be applied to a fluidic stream exiting the outlet of a channel so as to pass through the fluid in the general direction of fluid flow. The fluid may assume a surface charge which is susceptible to the electric field, and the electric field thereby applies an attractive force to the fluid in the direction of fluid flow, thus forming an approximate cone shape with a cross-sectional dimension of the fluidic stream decreasing in the direction of fluid flow.

In certain embodiments, the electric field applied to the fluid is at least about 0.01 V/micrometer, and, in some cases, at least about 0.03 V/micrometer, at least about 0.05 V/micrometer, at least about 0.08 V/micrometer, at least about 0.1 V/micrometer, at least about 0.3 V/micrometer, at least about 0.5 V/micrometer, at least about 0.7 V/micrometer, at least about 1 V/micrometer, at least about 1.2 V/micrometer, at least about 1.4 V/micrometer, at least about 1.6 V/micrometer, or at least about 2 V/micrometer. In some embodiments, even higher electric field intensities may be used, for example, at least about 2 V/micrometer, at least about 3 V/micrometer, at least about 5 V/micrometer, at least about 7 V/micrometer, or at least about 10 V/micrometer or more.

According to certain aspects of the invention, a first fluid and a second fluid are brought into contact, and sometimes mixed, within an article containing one or more channels or microfluidic channels, prior to spray drying. The first fluid and the second fluid can be miscible or immiscible. For example, the fluids may be immiscible within the time frame of formation of a stream of fluids (e.g., forming droplets), or within the time frame of reaction or interaction within the channel. As used herein, two fluids are “immiscible,” or not miscible, with each other when one is not soluble in the other to a level of at least 10% by weight at the temperature and under the conditions at which the fluids are exposed to each other.

The fluids may be hydrophilic or hydrophobic. For example, in one set of embodiments, a first fluid may be hydrophilic and a second fluid may be hydrophobic, a first fluid may be hydrophobic and a second fluid may be hydrophilic, or both fluids may each be hydrophilic or hydrophobic, etc. More than two fluids can be used in some embodiments. A hydrophobic fluid is generally immiscible in pure water while a hydrophilic fluid is generally miscible in pure water (of course, water is miscible in itself, and thus, water is a hydrophilic fluid).

As used herein, the term “fluid” generally refers to a substance that tends to flow and to conform to the outline of its container. Typically, fluids are materials that are unable to withstand a static shear stress, and when a shear stress is applied, the fluid experiences a continuing and permanent distortion. The fluid can have any suitable viscosity that permits at least some flow of the fluid. Non-limiting examples of fluids include liquids and gases, but may also include free-flowing solid particles, viscoelastic materials, and the like.

In some cases, one or more of the fluids within the article contain a species such as chemical, biochemical, or biological entities, cells, particles, beads, gases, molecules, pharmaceutical agents, drugs, DNA, RNA, proteins, fragrance, reactive agents, biocides, fungicides, preservatives, chemicals, or the like. Thus, the species can be any substance that can be contained in a fluid and can be differentiated from the fluid containing the species. For example, the species may be dissolved or suspended in the fluid. The species may be present in one or more of the fluids. If the fluids contain droplets, the species can be present in some or all of the droplets. Additional non-limiting examples of species that may be present include, for example, biochemical species such as nucleic acids such as siRNA, RNAi and DNA, proteins, peptides, or enzymes. Still other examples of species include, but are not limited to, nanoparticles, quantum dots, fragrances, proteins, indicators, dyes, fluorescent species, chemicals, or the like. As yet another example, the species may be a drug, pharmaceutical agent, or other species that has a physiological effect when ingested or otherwise introduced into the body, e.g., to treat a disease, relieve a symptom, or the like. In some embodiments, the drug may be a small-molecule drug, e.g., having a molecular weight of less than about 1000 Da or less than about 2000 Da.

In some aspects, a first fluid contains a species dissolved therein, where the species is insoluble (or soluble to a lesser degree) in a second fluid. Upon contact or mixing of the first fluid and the second fluid, the species is no longer able to remain dissolved (e.g., at the same concentration as before), and thus begins to precipitate. In some cases, such precipitation may occur within a channel, e.g., a channel used to expel fluids contained therein into the drying region, e.g., through an opening within the channel. Thus, a channel can contain a precipitating species in certain embodiments, e.g., after the first fluid and the second fluid come into contact.

In some cases, the precipitating species may deposit or “foul” one or more walls defining the channel. As discussed herein, various techniques may be used to reduce and/or eliminate fouling of the walls of the channels from occurring, for example, due to the use of channels having a relatively large cross-sectional aspect ratio, the use of one or more fluids to surround the fluid containing the precipitating species, the use of specific coatings on one or more walls of the channel, the control of fluid flow rates within the channel, or the like, or combinations of these and/or other techniques. Other examples of such techniques to prevent a first fluid or a species contained therein from contacting the walls defining the channel containing the first fluid are also discussed herein.

In some embodiments, as discussed below, the time of contact or the time of mixing of the first fluid and the second fluid may be kept relatively short, e.g., to control the amount of time in which the species is able to precipitate to form a solid. For instance, for relatively short times, particles such as microparticles or nanoparticles can be formed during the spray drying process, and in some cases, the size, the morphology, etc. of such particles may be controlled, as is discussed herein. In certain embodiments, mixing of the first fluid and the second fluid is controlled such that a precipitating species does not contact a wall of the channel.

Examples of such systems include those associated with liquid antisolvent precipitation (“LASP”). In general, in LASP, a first solution containing a solute dissolved in a solvent is mixed with an “antisolvent,” which causes supersaturation and/or precipitation of particles of solute. Typically, the solute is not soluble in the antisolvent, or at least has a relatively low degree of solubility within the second liquid. For example, the solubility of the solute in the antisolvent may be at least 1, 2, 3, 4, or 5 orders of magnitude (powers of 10) lower than the solubility of the solute in the solvent. Without wishing to be bound by any theory, it is believed that precipitation therein occurs via nucleation and/or growth by coagulation or condensation. In some cases, uniform mixing conditions may be used to ensure rapid and uniform supersaturation. Examples of potential solutes include, but are not limited to, danazol, ibuprofen, itraconazole, ascorbyl palmitate, fenofibrate, griseofulvin, and sulfamethoxazole. Non-limiting examples of solvents include, for example, acetone, dimethyl sulfoxide, tetrahydrofuran, ethanol, or isopropyl alcohol. The antisolvent may be, for example, water, an aqueous solution (e.g., a solution comprising water as a solvent, such as saline), or the like. As one example, the antisolvent may be a liquid that is miscible in water.

A specific non-limiting example of such a system is danazol (17 alpha-ethinyl testosterone) in isopropyl alcohol and water. Danazol has generally good solubility in isopropyl alcohol, but relatively poor solubility in water. Thus, in one set of embodiments, danazol is dissolved in isopropyl alcohol to form the first fluid, which is then contacted with water as the second fluid, e.g., within a channel such as a microfluidic channel. If flow within the channel is laminar, no substantial mixing of the isopropyl alcohol and water occurs within the channel (other than due to diffusion), and thus, the danazol generally remains dissolved within the isopropyl alcohol while contained within the channel, without substantially precipitating. However, once mixing of the first fluid and the second fluid occurs (e.g., due to the introduction of air into the channel to cause such mixing to occur), danazol is unable to remain dissolved in the mixed fluids, and thus precipitates to form a solid. In some cases, this mixing process may occur relatively rapidly, e.g., just before the fluids are expelled through an opening or a nozzle into a drying region. Accordingly, the danazol precipitates while the fluids are expelled to form droplets which dries while in the drying region. In such a manner, solid particles containing danazol can be formed by spray drying. In some cases, as discussed herein, control of the formation of such particles may be controlled, e.g., to produce relatively monodisperse particles, and/or relatively amorphous particles. For example, in certain embodiments, controlled generation of particles, such as amorphous particles, can be produced having relatively narrow size distribution and/or low mean particle sizes.

According to some embodiments of the invention, the time of exposure of a first fluid and a second fluid (e.g., a solvent and an antisolvent in the case of LASP) is kept relatively short. For instance, the first fluid and the second fluid may be kept separate, then brought into contact within a channel, such as a microfluidic channel, within an article. In some cases, the fluids may be brought into contact immediately before the fluids are expelled from a channel into a drying region in a spray dryer. For example, a first fluid and a second fluid can be brought into contact, and optionally mixed, in a channel having one or more openings that are used to expel the fluids into the drying region.

As mentioned, in some embodiments, the time of physical contact of the fluids prior to expulsion into the drying region may be relatively short, For example, the time of physical contact between the two fluids within a channel may be less than about 5 minutes, less than about 3 minutes, less than about 1 minute, less than about 30 seconds, less than about 20 seconds, less than about 15 seconds, less than about 10 seconds, less than about 8 seconds, less than about 6 seconds, less than about 5 seconds, less than about 4 seconds, less than about 3 seconds, less than about 2 seconds, less than about 1 second, less than about 0.5 seconds, less than about 0.3 seconds, less than about 0.2 seconds, or less than about 0.1 seconds.

As a specific non-limiting example, referring now to fluidic system 10 in FIG. 8, a first fluid in first channel 20 from first fluid source 25 may be contacted at junction 23 by a second fluid from second channel 30 (and/or third channel 32) from second fluid source 35, and the two fluids delivered to nozzle 12 to be expelled into drying region 15. In some cases, e.g., depending on the fluid flow rates within channel first channel 20, and second channel 30 and/or third channel 32, the time of contact of the two fluids may be relatively short, for example, as described above.

In some embodiments, the amount of time of physical contact of the fluids within the article can be controlled using various elements within the article, such as the use of chambers (e.g., to allow mixing to occur) and/or channel geometries (e.g., having different dimensions, sizes, lengths, cross-sectional areas, shapes, or the like). As a specific non-limiting example, in one set of embodiments, one or more “meandering” channels may be used to control the amount of time of physical contact. Meandering channels may have any suitable size and or shape, but are essentially longer channels used to increase the time of physical contact of the fluids due to their increased length. For instance, the meandering channel can have a zigzag profile, or another suitable geometry. The length of a suitable meandering channel may depend on various factors such as the fluids to be brought into contact, the desired length of exposure, and the flow rates of the fluids within the channel.

In one set of embodiments, fluid mixing with the channels, e.g., of a first fluid and a second fluid, is controlled using bolus flow. Under bolus flow, a relatively large object, such as a large droplet or a particle, may substantially fill a channel (e.g., the cross-section of the channel may be substantially or completely encompassed by the droplet). A series of boluses within the channel can delineate or partition the flow of fluid within the channel between the boluses into individual segments, and fluid within the individual segments may recirculate or otherwise mix. In certain embodiments, the material forming the bolus may be immiscible or be substantially insoluble in one or more of the fluids between the boluses, although in other embodiments, the bolus material can be miscible or soluble.

The boluses, in some cases, may substantially fill a channel such that, in a cross-sectional plane of the channel, at least about 50% of the channel is filled by the bolus material (e.g., a solid, a liquid, a gas, etc.). In certain instances, at least about 60%, at least about 70%, at least about 80%, at least about 90%, at least about 95%, at least about 97%, at least about 99%, or the entire cross-sectional plane of the channel is filled with the bolus material. In some embodiments, the boluses may delineate or partition the fluid within the channel such that the volume of the delineated portions is no more than about 10 ml, no more than about 3 ml, no more than about 1 ml, no more than about 300 microliters, no more than about 100 microliters, no more than about 30 microliters, no more than about 10 microliters, no more than about 3 microliters, no more than about 1 microliter, no more than about 300 nl, no more than about 100 nl, no more than about 30 nl, no more than about 10 nl, no more than about 3 nl, or no more than about 1 nl.

In some embodiments, the boluses may be separated on a substantially repeating basis, e.g., such that, for a given location within the channel, the boluses pass the location at an average frequency of less than about 100 Hz, less than about 50 Hz, less than about 30 Hz, less than about 10 Hz, less than about 5 Hz, less than about 3 Hz, less than about 1 Hz, less than about 0.5 Hz, less than about 0.3 Hz, or less than about 0.1 Hz (i.e., where 0.1 Hz is equivalent to one bolus passing a given location every 10 s). In some embodiments, the boluses may have a volume, on average, of no more than about 10 ml, no more than about 3 ml, no more than about 1 ml, no more than about 300 microliters, no more than about 100 microliters, no more than about 30 microliters, no more than about 10 microliters, no more than about 3 microliters, no more than about 1 microliter, no more than about 300 nl, no more than about 100 nl, no more than about 30 nl, no more than about 10 nl, no more than about 3 nl, or no more than about 1 nl.

In some cases, such mixing (e.g., of fluids within a channel, including within segments between boluses, or the like) may further be enhanced by controlling the geometry of the channel. For example, the fluid may be passed through one or more channels or other systems which cause the fluid to change its velocity and/or direction of movement. The change of direction may alter convection patterns within the fluid, causing or enhancing mixing within the fluid between the boluses. For instance, the channel may contain chambers, one or more bends or zigzags, expansion regions, constriction regions, valves, etc., and/or any other suitable combination of these and/or other channel elements. Other examples of channel geometries used to control mixing, e.g., within droplets and/or other fluids contained within a channel, may be seen in U.S. patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007; each of which is incorporated herein by reference in its entirety.

In some embodiments, the fluids flow through the channel at relatively high flow rates or speeds, for example, to achieve contact times such as those described herein. The flow within the channels can be laminar or turbulent. In some cases, flow through the channel occurs such that the Reynolds number of the flow is at least about 0.001, at least about 0.003, at least about 0.005, at least about 0.01, at least about 0.03, at least about 0.05, at least about 0.1, at least about 0.3, or at least about 0.5. Higher Reynolds numbers may be used in other embodiments (e.g., corresponding to turbulent flow), for instance, Reynolds numbers of at least about 1, at least about 3, at least about 5, at least about 10, at least about 30, at least about 50, at least about 100, at least about 300, at least about 500, or at least about 1000. In still other embodiments, however, flow through the channel may occur such that the Reynolds number of the flow is less than 1000, less than about 300, less than about 100, less than about 30, less than about 10, less than about 3, or less than about 1. In yet other embodiments of the invention, the volumetric flow rate of fluid through the channel may be at least about 0.01 ml/h at least about 0.03 ml/h, at least about 0.05 ml/h, at least about 0.1 ml/h, at least about 0.3 ml/h, at least about 0.5 ml/h, at least about 1 ml/h, at least about 3 ml/h, at least about 5 ml/h, at least about 10 m/l, at least about 30 ml/h, at least about 50 ml/h, or at least about 100 ml/h.

Relatively high flow rates may be achieved, for example, by increasing or controlling the difference in pressure between one or more of the fluid sources within the article containing channels, and the pressure within the drying region of the spray dryer. For example, the pressure within the drying region may be at ambient pressure (approximately 1 atm), and/or the pressure may be higher or lower. As specific non-limiting examples, the pressure within the drying region may be less than about 50 mmHg, less than about 100 mmHg, less than about 150 mmHg, less than about 200 mmHg, less than about 250 mmHg, less than about 300 mmHg, less than about 350 mmHg, less than about 400 mmHg, less than about 450 mmHg, less than about 500 mmHg, at least 550 mmHg, at least 600 mmHg, at least 650 mmHg, less than about 700 mmHg, or less than about 750 mmHg below atmospheric pressure. As another example, the pressure of one or more of the fluid sources within the article may be at least about 1 bar, at least about 1.1 bars, at least about 1.2 bars, at least about 1.3 bars, at least about 1.4 bars, at least about 1.5 bars, at least about 1.7 bars, at least about 2 bars, at least about 2.5 bars, at least about 3 bars, at least about 4 bars, at least about 5 bars, etc.

Upon being expelled from the channel into a suitable drying region, the fluids can condense to form individual or discrete droplets within the drying region in certain embodiments, for instance, due to surface tension or other effects. Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet. The droplets may be formed steadily, for example, forming a steady or linear stream of droplets, or in other embodiments, larger numbers of droplets may be formed, for example, creating a mist or a spray of individual droplets, e.g., within the drying region.

In some cases, the fluids are expelled from the channel such that relatively small droplets are formed, for instance, such that the average diameter of the droplets that are formed is less than about 1 cm. In certain embodiments, as non-limiting examples, the average diameter of the droplets can also be less than about 1 mm, less than about 500 micrometers, less than about 200 micrometers, less than about 100 micrometers, less than about 75 micrometers, less than about 50 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the droplets may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases. The “average diameter” of a population of droplets is the arithmetic average of the diameters of the droplets.

In certain embodiments, the fluid droplets within the drying region, e.g., after being expelled from a channel, may be substantially monodisperse. For example, the fluid droplets may have a distribution in diameters such that at least about 50%, at least about 60%, at least about 70%, about 80%, at least about 85%, at least about 90%, at least about 95%, at least about 97%, or at least about 99% of the droplets have a diameter that is no more than about 10% different, no more than about 7% different, no more than about 5% different, no more than about 4% different, no more than about 3% different, no more than about 2% different, or no more than about 1% different from the average diameter of the droplets.

In some cases, at least a portion of the fluids within the individual droplets may harden or solidify, e.g., within a drying region. For example, some of the droplets, and/or a portion of some of the droplets, can harden to form particles. The particles can then be subsequently collected. The particles may comprise, for example, the hardened first fluid, the hardened second fluid, and/or a species contained in a fluid that is hardened, depending on the material that is evaporated or driven off (e.g., water, and/or other volatile fluids) within the drying region from the fluidic droplets. If more than one material is present, the material can be homogenously or heterogeneously distributed within the particles. The particles may, in some embodiments, have substantially the same shape and/or be substantially the same size as the fluidic droplets. For example, the particles can be monodisperse, e.g., as discussed above, and/or the particles may be spherical, or non-spherical in certain cases. In some cases, some or all of the particles may be microparticles and/or nanoparticles. Microparticles generally have an average diameter of less than about 1 mm (e.g., such that the average diameter of the particles is typically measured in micrometers), while nanoparticles generally have an average diameter of less than about 1 micrometer (e.g., such that the average diameter of the particles is typically measured in nanometers). In some cases, the particles may have a distribution in diameters such that at least about 50%, at least about 60%, at least about 70%, about 80%, at least about 85%, at least about 90%, at least about 95%, at least about 97%, or at least about 99% of the droplets have a diameter that is no more than about 10% different, no more than about 7% different, no more than about 5% different, no more than about 4% different, no more than about 3% different, no more than about 2% different, or no more than about 1% different from the average diameter of the particles.

In one set of embodiments, the particles include a species contained within a fluid used to form the particles. For example, a first fluid may contain a species, optionally mixed with a second fluid, and the mixture of the first and second fluid expelled to form fluid droplets within the drying region. The first and/or second fluids can be evaporated or driven off in the drying region, thereby causing the species to form solid particles within the drying region. The solid particles may be crystalline, or amorphous in certain embodiments, for example, depending on the amount of time the droplets or particles are exposed to the drying region and the speed at which the droplets dry and/or solidify to form particles. As a specific non-limiting example, relatively short times in which a species is precipitated can be useful to cause amorphous particles to form. For instance, if precipitation of a species occurs due to an interaction between a first fluid and a second fluid, then the time of physical contact of the fluids prior to expulsion into the drying region can be kept relatively short to facilitate amorphous particle formation.

The degree of crystallinity of a particle can be determined using any technique known to those of ordinary skill in the art, for example, X-ray diffraction (XRD) techniques. In some applications, amorphous particles may be desirable since the particles typically will dissolve more quickly than similar crystalline particles. For example, if the particles are used as drugs, an amorphous particle may exhibit significantly increased bioavailability, e.g., as compared to similar crystalline particles. In some embodiments, the particles can exhibit a degree of crystallinity that is between completely crystalline and completely amorphous. For example, the particles can exhibit an average degree of crystallinity (mass of the particle that is crystalline versus the total mass of the particle) of less than about 90%, less than about 80%, less than about 70%, less than about 60%, less than about 50%, less than about 40%, less than about 30%, less than about 20%, or less than about 10%. In some cases, the particles may exhibit an average degree of crystallinity that is at least about 10%, at least about 20%, at least about 30%, at least about 40%, at least about 50%, at least about 60%, at least about 70%, at least about 80%, or at least about 90%.

In certain embodiments, the droplet may also contain a crystallization inhibitor, e.g., present within the first and/or second fluids, and/or introduced separately. For example, the crystallization inhibitor can reduce and/or eliminate crystallization within the fluidic droplet as the fluidic droplet dries, thereby causing the particle to become amorphous, or at least have less crystallinity and a greater amount of amorphous character. Any suitable crystallization inhibitor can be used, depending on the species and/or fluids being solidified. As a specific non-limiting example, the crystallization inhibitor may be a polymer such as poly(vinylpyrrolidone), which does not readily crystallize (at least under conditions in which the particles are formed), and which in some embodiments is able to reduce or inhibit crystal growth in pharmaceutical formulations, e.g., comprising danazol, ibuprofen, or other suitable species such as those described herein.

Certain aspects of the invention are generally directed to techniques for scaling up or “numbering up” devices such as those discussed herein. For example, in one set of embodiments, a channel can have more than one opening or nozzle, which may be used to expel a plurality of droplets into a drying region or into more than one drying region. As another example, an article may contain more than one channel, which may be used to expel a plurality of droplets into a drying region or into more than one drying region.

For instance, an article can contain at least 2 channels, at least 3 channels, at least 5 channels, at least 10 channels, at least 25 channels, at least 50 channels, at least 100 channels, some or all of which channels may have on or more openings or nozzles. As yet another example, more than one article may be present, some or all of which may have at least one opening through which droplets are expelled, for instance, into a drying region or into more than one drying region. As still another example, combinations of any of these may be present.

If more than one article is present, the articles may independently be substantially the same or different. In some embodiments, for instance, greater production of droplets or particles can be achieved simply by adding additional substantially identical copies of the articles used to produce droplets. For example, a spray dryer may contain at least 2 articles, at least 3 articles, at least 5 articles, at least 10 articles, at least 25 articles, at least 50 articles, at least 100 articles, at least 250 articles, at least 500 articles, at least 1000 articles, etc., which may be used to expel a plurality of droplets into a drying region or into more than one drying region. The articles can draw fluids from a common fluid source or more than one common fluid source in some embodiments. In certain embodiments, for example, each article can have its own fluid source.

Those of ordinary skill in the art will be aware of other techniques useful for scaling up or numbering up devices or articles such as those discussed herein. For example, in some embodiments, a fluid distributor can be used to distribute fluid from one or more inputs to a plurality of outputs, e.g., in one more devices. For instance, a plurality of articles may be connected in three dimensions. In some cases, channel dimensions are chosen that allow pressure variations within parallel devices to be substantially reduced. Other examples of suitable techniques include, but are not limited to, those disclosed in International Patent Application No. PCT/US2010/000753, filed Mar. 12, 2010, entitled “Scale-up of Microfluidic Devices,” by Romanowsky, et al., published as WO 2010/104597 on Nov. 16, 2010, incorporated herein by reference in its entirety.

Additional aspects of the invention generally relate to systems and methods for manipulating droplets within a channel contained within an article, e.g., prior to expelling the droplets into a drying region. Non-limiting examples of droplet manipulation include creating droplets, splitting droplets, fusing droplets, mixing within droplets, screening droplets, sorting droplets, etc., some of which are discussed herein. Further non-limiting examples of techniques for manipulating droplets may be seen in various documents that are incorporated herein by reference.

For example, in some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Certain embodiments are generally directed to systems and methods for splitting a droplet into two or more droplets. For example, a droplet can be split using an applied electric field. The droplet may have a greater electrical conductivity than the surrounding liquid, and, in some cases, the droplet may be neutrally charged. In certain embodiments, in an applied electric field, electric charge may be urged to migrate from the interior of the droplet to the surface to be distributed thereon, which may thereby cancel the electric field experienced in the interior of the droplet. In some embodiments, the electric charge on the surface of the droplet may also experience a force due to the applied electric field, which causes charges having opposite polarities to migrate in opposite directions. The charge migration may, in some cases, cause the drop to be pulled apart into two separate droplets.

Some embodiments of the invention generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

Certain embodiments of the invention are also related to systems and methods for allowing the mixing of more than one fluid to occur within a droplet. For example, in various embodiments, two or more droplets may be allowed to fuse or coalesce, and the two or more fluids from the two or more original droplets allowed to mix. It should be noted that when two droplets fuse or coalesce, perfect mixing within the droplet does not instantaneously occur. The mixing may occur through natural means, for example, through diffusion (e.g., through the interface between the regions), through reaction of the fluids with each other, through fluid flow within the droplet (i.e., convection), etc. In some cases, mixing can be enhanced through certain systems external of the droplet. For example, a droplet can be passed through one or more channels, channel elements, bends, zigzags, valves, etc. which can cause the droplet to change its velocity and/or direction of movement. The change of direction may alter convection patterns within the droplet, causing the fluids to be at least partially mixed.

In one set of embodiments, a fluid may be injected into a droplet, which may cause mixing of the injected fluid with the other fluids within the droplet to occur. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, incorporated herein by reference in its entirety.

Yet other embodiments of the invention are generally directed to systems and methods for screening or sorting droplets, and in some cases, at relatively high rates. For example, a characteristic of a droplet may be sensed and/or determined in some fashion (e.g., as further described below), then the droplet may be directed towards a particular region of the device, for example to be expelled into a drying region, or rejected from further processing or manipulation, or sent to waste. For example, a characteristic of a droplet may be sensed and/or determined in some fashion, for example, as described herein (e.g., fluorescence of the droplet may be determined), and, in response, an electric field may be applied or removed from the droplet to direct the droplet to a particular region (e.g. a channel for expulsion into a drying region). In some cases, high sorting speeds may be achievable using certain systems and methods of the invention.

In one set of embodiments, a droplet can be directed by creating an electric charge (e.g., as previously described) on the droplet, and steering the droplet using an applied electric field, which may be an AC field, a DC field, etc. As an example, an electric field can be selectively applied and removed (or a different electric field may be applied, e.g., a reversed electric field) as needed to direct the droplet to a particular region, for instance, within an article. The electric field may be selectively applied and removed as needed, in some embodiments, without substantially altering the flow of the liquid in the channel containing the droplet. For example, a liquid may flow on a substantially steady-state basis or other predetermined basis through a channel, and droplets contained within the liquid may be directed to various regions, e.g., using an electric field, without substantially altering the flow of the liquid through the fluidic system.

In another set of embodiments, a droplet may be sorted or steered by inducing a dipole in the droplet (which may be initially charged or uncharged), and sorting or steering the droplet using an applied electric field. The electric field can be an AC field, a DC field, etc. In other embodiments, however, the droplets may be screened or sorted within a fluidic system of the invention by altering the flow of the liquid containing the droplets. For instance, in one set of embodiments, a droplet is steered or sorted by directing the liquid surrounding the droplet into a first channel, a second channel, etc.

In still another set of embodiments, pressure within a fluidic system, for example, within different channels or within different portions of a channel, can be controlled to direct the flow of droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a droplet into a channel, e.g., by causing directed movement of the liquid containing the droplet. Non-limiting examples of devices able to cause the expansion and/or contraction of a liquid reservoir include pistons and piezoelectric components.

In certain embodiments of the invention, sensors are provided that can sense and/or determine one or more characteristics of the droplets, and/or a characteristic of a portion of the channel containing the droplet (e.g., the liquid surrounding the droplet, the article containing the channel, etc.) in such a manner as to allow the determination of one or more characteristics of the droplets. Characteristics determinable with respect to the droplet and usable in the invention can be identified by those of ordinary skill in the art. Non-limiting examples of such characteristics include fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), or the like. In some cases, the sensor may be connected to a processor, which in turn, cause an operation or a manipulation to be performed on the droplet, for example, as discussed herein.

As another example, a sensor may be in sensing communication with the droplet and/or the portion of the channel containing the droplet fluidly, optically or visually, thermally, pneumatically, electronically, or the like. The sensor can be positioned in the article e.g., proximate the channel, or positioned separately from the article but with physical, electrical, and/or optical communication with the channel. For instance, a sensor may be free of any physical connection with a channel containing a droplet, but may be positioned so as to detect electromagnetic radiation arising from the droplet or the channel, such as infrared, ultraviolet, or visible light. The electromagnetic radiation may be produced by the droplet, and/or may arise from other portions of the channel (or externally of the channel or article) and interact with the fluidic droplet and/or the portion of the channel containing the fluidic droplet in such as a manner as to indicate one or more characteristics of the fluidic droplet, for example, through absorption, reflection, diffraction, refraction, fluorescence, phosphorescence, changes in polarity, phase changes, changes with respect to time, etc. Non-limiting examples of sensors useful in the invention include optical or electromagnetically-based systems. For example, the sensor may be a fluorescence sensor (e.g., stimulated by a laser), a microscopy system (which may include a camera or other recording device), or the like. As another example, the sensor may be an electronic sensor, e.g., a sensor able to determine an electric field or other electrical characteristic. For example, the sensor may detect capacitance, inductance, etc., of a fluidic droplet and/or the portion of the channel containing the fluidic droplet.

Other aspects of the present invention include the following. Certain embodiments of the present invention present a versatile tool, e.g., for the development of new formulations. For example, small quantities of a drug, pharmaceutical agent, or other species can be tested in some cases. In certain embodiments, for instance, a drug, pharmaceutical agent, or other species may be tested for its spray drying characteristics relatively rapidly, and/or without requiring a large initial amount of sample for testing purposes. Conditions for spray drying may be changed relatively rapidly, e.g., before and/or during spray drying experiments, in order to experiment or optimize various formulations, and in some cases without requiring a relatively large amount of drug, pharmaceutical agent, or other species. For instance, no more than about 100 g, no more than about 50 g, no more than about 30 g, no more than about 10 g, no more than about 5 g, no more than about 3 g, no more than about 1 g, no more than about 500 mg, no more than about 300 mg, or no more than about 100 mg of drug, pharmaceutical agent, or other species may be used in the spray dryer in certain embodiments, e.g., to produce particles. In some cases, relatively small numbers or masses of particles may be produced in a given spray drying experiment, e.g., allowing conditions to be rapidly changed, for example, as discussed above. For instance, no more than about 100 g, no more than about 50 g, no more than about 30 g, no more than about 10 g, no more than about 5 g, no more than about 3 g, no more than about 1 g, no more than about 500 mg, no more than about 300 mg, or no more than about 100 mg of particles or solids may be formed using the spray dryer. In some embodiments, particles having tunable compositions may be prepared, e.g., as discussed herein. In some cases, the composition of the particles may be easily controlled, e.g., by controlling fluid flow into the spray dryer.

In addition, in some embodiments, a spray dryer may have a relatively low dead volume, which may thus reduce waste of sample and/or facilitate experiments that use minimal amounts of drugs, pharmaceutical agents, or other species, such as is described herein. The dead volume of the spray dryer includes volumes within the spray dryer which contain volumes of fluid that are not able to be expelled by the spray dryer into the drying region during normal operation of the spray dryer.

In some cases, a suspension may be produced using spray dryers such as those discussed herein. Such suspensions may be used, for example, to enhance the dissolution rate and bioavailability of hydrophobic drugs. For instance, a suspension can be prepared by spraying a fluid into a carrier liquid. In some embodiments, the carrier liquid may contain a stabilizer or a surfactant, e.g., as in a solution. In other embodiments, however, no stabilizer or surfactant may be present in the carrier liquid. In some cases, the fluid being expelled may be dried sufficiently to produce particles prior to contacting the carrier liquid; in other cases, however, the fluids may enter the solution not fully dried, for example, to form a liquid suspension in the carrier liquid.

In addition, in some embodiments, a spray dryer may be directly connected to a vial, a sample holder, an ampoule, etc., without necessarily requiring intermediate processing and/or storage, for example, fluid transport or filling from a collection chamber to a vial, which can cause waste, alteration of physical or chemical properties, etc. For example, one or more relatively small vials (or other collection chambers) may be used to directly collect material produced by the spray dryer. The vial or other collection chamber may have a relatively small volume, e.g., less than about 100 ml, less than about 50 ml, less than about 30 ml, less than about 20 ml, less than about 15 ml, less than about 10 ml, less than about 5 ml, etc. In some cases, one collection chamber is used, although in other cases, more than one may be used, e.g., such that one is replaced by the next (manually or automatically) after a certain time and/or after a certain amount has been collected therein.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al).

In one set of embodiments, various structures or components of the articles described herein can be formed of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference).

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material.

In some embodiments, various structures or components of the article are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, waxes, metals, or mixtures or composites thereof heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, MI, and particularly Sylgard 182, Sylgard 184, and Sylgard 186. Silicone polymers including PDMS have several beneficial properties simplifying fabrication of various structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Thus, in certain embodiments, the design and/or fabrication of the spray dryer may be relatively simple, e.g., by using relatively well-known soft lithography and other techniques such as those described herein. In addition, in some embodiments, rapid and/or customized design of the spray dryer is possible, for example, in terms of geometry. In one set of embodiments, the spray dryer may be produced to be disposable, for example, in embodiments where the spray dryer is used with substances that are radioactive, toxic, poisonous, reactive, biohazardous, etc., and/or where the profile of the substance (e.g., the toxicology profile, the radioactivity profile, etc.) is unknown.

Another advantage to forming channels or other structures (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

In some embodiments, one or more walls or portions of a channel may be coated, e.g., with a coating material, including photoactive coating materials. The coating materials can be used in certain instances to control and/or alter the hydrophobicity of the wall of a channel. In some embodiments, a sol-gel is provided that can be formed as a coating on a substrate such as the wall of a channel such as a microfluidic channel. One or more portions of the sol-gel can be reacted to alter its hydrophobicity, in some cases. For example, a portion of the sol-gel may be exposed to light, such as ultraviolet light, which can be used to induce a chemical reaction in the sol-gel that alters its hydrophobicity. The sol-gel may include a photoinitiator which, upon exposure to light, produces radicals. Optionally, the photoinitiator is conjugated to a silane or other material within the sol-gel. The radicals so produced may be used to cause a condensation or polymerization reaction to occur on the surface of the sol-gel, thus altering the hydrophobicity of the surface. In some cases, various portions may be reacted or left unreacted, e.g., by controlling exposure to light (for instance, using a mask).

Thus, in one aspect of the invention, a coating on the wall of a channel may be a sol-gel. As is known to those of ordinary skill in the art, a sol-gel is a material that can be in a sol or a gel state. In some cases, the sol-gel material may comprise a polymer.

The sol state may be converted into the gel state by chemical reaction. In some cases, the reaction may be facilitated by removing solvent from the sol, e.g., via drying or heating techniques. Thus, in some cases, e.g., as discussed below, the sol may be pretreated before being used, for instance, by causing some condensation to occur within the sol. Sol-gel chemistry is, in general, analogous to polymerization, but is a sequence of hydrolysis of the silanes yielding silanols and subsequent condensation of these silanols to form silica or siloxanes.

In some embodiments, the sol-gel coating may be chosen to have certain properties, for example, having a certain hydrophobicity. The properties of the coating may be controlled by controlling the composition of the sol-gel (for example, by using certain materials or polymers within the sol-gel), and/or by modifying the coating, for instance, by exposing the coating to a condensation or polymerization reaction to react a polymer to the sol-gel coating, as discussed herein.

For example, the sol-gel coating may be made more hydrophobic by incorporating a hydrophobic polymer in the sol-gel. For instance, the sol-gel may contain one or more silanes, for example, a fluorosilane (i.e., a silane containing at least one fluorine atom) such as heptadecafluorosilane or heptadecafluorooctylsilane, or other silanes such as methyltriethoxy silane (MTES) or a silane containing one or more lipid chains, such as octadecylsilane or other CH₃(CH₂)ₙ— silanes, where n can be any suitable integer. For instance, n may be greater than 1, 5, or 10, and in some cases, less than about 20, 25, or 30. The silanes may also optionally include other groups, such as alkoxide groups, for instance, octadecyltrimethoxysilane. Other examples of suitable silanes include alkoxysilanes such as ethoxysilane or methoxysilane, halosilanes such as chlorosilanes, or other silicon-containing compounds containing hydrolyzable moieties on the silicon atom, such as hydroxide moieties. In general, most silanes can be used in the sol-gel, with the particular silane being chosen on the basis of desired properties such as hydrophobicity. Other silanes (e.g., having shorter or longer chain lengths) may also be chosen in other embodiments of the invention, depending on factors such as the relative hydrophobicity or hydrophilicity desired. In some cases, the silanes may contain other groups, for example, groups such as amines, which would make the sol-gel more hydrophilic. Non-limiting examples include diamine silane, triamine silane, or N-[3-(trimethoxysilyl)propyl]ethylene diamine silane. The silanes can be reacted to form networks within the sol-gel, and the degree of condensation may be controlled by controlling the reaction conditions, for example by controlling the temperature, amount of acid or base present, or the like.

In some cases, more than one silane is present in the sol-gel. For instance, the sol-gel can include fluorosilanes to cause the resulting sol-gel to exhibit greater hydrophobicity, and other silanes (or other compounds) that facilitate the production of polymers. In some cases, materials able to produce SiO₂ compounds to facilitate condensation or polymerization may be present, for example, TEOS (tetraethyl orthosilicate). In some embodiments, the silane may have up to four chemical moieties bonded to it, and in some cases, one of the moieties may be on RO— moiety, where R is an alkoxide or other chemical moieity, for example, so that the silane can become incorporated into a metal oxide-based network. In addition, in some cases, one or more of the silanes can be hydrolyzed to form the corresponding silanol.

In addition, it should be understood that the sol-gel is not limited to containing only silanes, and other materials may be present in addition to, or in place of, the silanes. For instance, the coating may include one or more metal oxides, such as SiO₂, vanadia (V205), titania (TiO₂), and/or alumina (Al₂O₃). As other examples, the sol-gel may comprise moieties containing double bonds, or otherwise are reactive within any polymerization reactions, for example, thiols for participation in radical polymerization.

The sol-gel may be present as a coating on the substrate, and the coating may have any suitable thickness. For instance, the coating may have a thickness of no more than about 100 micrometers, no more than about 30 micrometers, no more than about 10 micrometers, no more than about 3 micrometers, or no more than about 1 micrometer. Thicker coatings may be desirable in some cases, for instance, in applications in which higher chemical resistance is desired. However, thinner coatings may be desirable in other applications, for instance, within relatively small microfluidic channels.

In one set of embodiments, the hydrophobicity of the sol-gel coating can be controlled, for instance, such that a first portion of the sol-gel coating is relatively hydrophobic, and a second portion of the sol-gel coating is more or less relatively hydrophobic than the first portion. The hydrophobicity of the coating can be determined using techniques known to those of ordinary skill in the art, for example, using contact angle measurements such as those discussed herein. For instance, in some cases, a first portion of a substrate (e.g., within a microfluidic channel) can have a hydrophobicity that favors an organic solvent to water, while a second portion can have a hydrophobicity that favors water to the organic solvent.

The hydrophobicity of the sol-gel coating can be modified, for instance, by exposing at least a portion of the sol-gel coating to a condensation or polymerization reaction to react a polymer to the sol-gel coating. The polymer reacted to the sol-gel coating may be any suitable polymer, and may be chosen to have certain hydrophobicity properties. For instance, the polymer may be chosen to be more hydrophobic or more hydrophilic than the substrate and/or the sol-gel coating. As an example, a hydrophilic polymer that could be used is poly(acrylic acid).

The polymer may be added to the sol-gel coating by supplying the polymer in monomeric (or oligomeric) form to the sol-gel coating (e.g., in solution), and causing a condensation or polymerization reaction to occur between the polymer and the sol-gel. For instance, free radical polymerization may be used to cause bonding of the polymer to the sol-gel coating. In some embodiments, a reaction such as free radical polymerization may be initiated by exposing the reactants to heat and/or light, such as ultraviolet (UV) light, optionally in the presence of a photoinitiator able to produce free radicals (e.g., via molecular cleavage) upon exposure to light. Those of ordinary skill in the art will be aware of many such photoinitiators, many of which are commercially available, such as Irgacur 2959 (Ciba Specialty Chemicals), aminobenzophenone, benzophenone, or 2-hydroxy-4-(3-triethoxysilylpropoxy)-diphenylketone (51H6200.0, ABCR GmbH & Co. KG).

The photoinitiator may be included with the polymer added to the sol-gel coating, or in some cases, the photoinitiator may be present within the sol-gel coating. The photoinitiators can also be introduced within the sol-gel coating after the coating step, in some embodiments. As an example, a photoinitiator may be contained within the sol-gel coating, and activated upon exposure to light. The photoinitiator may also be conjugated or bonded to a component of the sol-gel coating, for example, to a silane. As an example, a photoinitiator such as Irgacur 2959 can be conjugated to a silane-isocyanate via a urethane bond (where a primary alcohol on the photoinitiator may participate in nucleophilic addition with the isocyanate group, which can produce a urethane bond).

Accordingly, some aspects of the present invention are generally directed to systems and methods for coating such a sol-gel onto at least a portion of a substrate. In one set of embodiments, a substrate, such as a microfluidic channel, is exposed to a sol, which is then treated to form a sol-gel coating. In some cases, the sol can also be pretreated to cause partial condensation or polymerization to occur. Extra sol-gel coating may optionally be removed from the substrate. In some cases, as discussed, a portion of the coating may be treated to alter its hydrophobicity (or other properties), for instance, by exposing the coating to a solution containing a monomer and/or an oligomer, and causing condensation or polymerization of the monomer and/or oligomer to occur with the coating.

The sol may be contained within a solvent, which can also contain other compounds such as photoinitiators including those described above. In some cases, the sol also comprises one or more silane compounds. The sol may be treated to form a gel using any suitable technique, for example, by removing the solvent using chemical or physical techniques, such as heat. For instance, the sol can be exposed to a temperature of at least about 50° C., at least about 100° C., at least about 150° C., at least about 200° C., or at least about 250° C., which may be used to drive off or vaporize at least some of the solvent. As a specific example, the sol may be exposed to a hotplate set to reach a temperature of at least about 200° C. or at least about 250° C., and exposure of the sol to the hotplate may cause at least some of the solvent to be driven off or vaporized. In some cases, however, the sol-gel reaction may proceed even in the absence of heat, e.g., at room temperature. Thus, for instance, the sol may be left alone for a while (e.g., about an hour, about a day, etc.), and/or air or other gases, or liquids, may be passed over the sol, to allow the sol-gel reaction to proceed.

In other embodiments, other techniques of initiation may be used instead of or in addition to photoinitiators. Examples inculde, but are not limited to, redox initiation, thermal decomposition triggered by e.g. heating portions of a device (e.g., this can be done by liquid streams that have a certain temperature or contain an oxidizing or a reducing chemical). In another embodiment, functionalization of the surfaces may be achieved by polyaddition and polycondensation reactions, for instance, if the surface contains reactive groups that can participate in the reaction. Silanes containing a desired functionality can also be added in some cases, e.g., silanes containing COOH moieties, NH₂ moieties, SO₃H moieties, SO₄H moieties, OH moieties, PEG-chains, or the like).

In some cases, any ungelled sol that is still present can be removed from the substrate. The ungelled sol may be actively removed, e.g., physically, by the application of pressure or the addition of a compound to the substrate, etc., or the ungelled sol may be removed passively in some cases. For instance, in some embodiments, a sol present within a microfluidic channel is heated to vaporize solvent, which builds up in a gaseous state within the microfluidic channels, thereby increasing pressure within the microfluidic channels. The pressure, in some cases, may be enough to cause at least some of the ungelled sol to be removed or “blown” out of the microfluidic channels.

In certain embodiments, a portion of the coating may be treated to alter its hydrophobicity (or other properties) after the coating has been introduced to the substrate. In some cases, the coating is exposed to a solution containing a monomer and/or an oligomer, which is then condensed or polymerized to bond to the coating, as discussed above. For instance, a portion of the coating may be exposed to heat or to light such as ultraviolet right, which may be used to initiate a free radical polymerization reaction to cause polymerization to occur. Optionally, a photoinitiator is present, e.g., within the sol-gel coating, to facilitate this reaction. In some embodiments, the photoinitiator can also contain double bonds, thiols, and/or other reactive groups such that the monomers and/or oligomers can be covalently linked to the sol-gel coating.

The following documents are incorporated herein by reference in their entireties: U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., now U.S. Pat. No. 7,708,949, issued May 4, 2010; U.S. patent application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009; and U.S. Patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007. Also incorporated herein by reference in their entireties are U.S. Provisional Patent Application Ser. No. 61/425,415, filed Dec. 21, 2010, entitled “Spray Drying Techniques,” by Abate, et al., and U.S. Provisional Patent Application Ser. No. 61/485,026, filed May 11, 2011, entitled “Spray Drying Techniques,” by Abate, et al.

The following examples are intended to illustrate certain embodiments of the present invention, but do not exemplify the full scope of the invention.

### EXAMPLE 1

Spraying drying is an important technique allowing drying of solutions, emulsions, or suspensions in one step. The final product may be a fine powder having a large surface. Pharmaceutical applications of spray drying techniques include a broad field ranging from, for example, manufacturing dry plant extracts avoiding decomposition of thermo-sensitive components, to the production of excipients for compression with improved binding characteristics. However, conventional spray dryer techniques often induce high production costs, as the fabrication process involves high pressures or complex experimental setups. In addition, particle sizes below 100 nm, as often required for targeted drug delivery, are usually not achievable with commercially available spray dryers.

As discussed in this example, these limitations can be overcome using microfluidics. One convenient technique to fabricate rather sophisticated microfluidic devices is soft lithography using polydimethylsiloxane (“PDMS”). However, hydrophobic compounds can adsorb onto PDMS microchannels and foul the device. An improved system for fabricating nanoparticles from hydrophobic drugs would combine the versatility of microfluidics with the ability to process hydrophobic drugs by spray drying.

Accordingly, this example is generally directed to the fabrication of hydrophobic drug nanoparticles using a microfluidc spray dryer. The device geometry using in this particular example has a high aspect ratio and is rendered hydrophilic by oxygen plasma treatment. This prevents the adsorption of hydrophobic precipitates on the channel walls, thus allowing the use of hydrophobic drugs in this PDMS-based microfluidic devices. By controlling the collection distance of the spray, the crystallinity of the product may also be controlled. Thus, this microfluidic device allows for the fabrication of drug nanoparticles of, e.g., less than 100 nm in diameter. This device also allows, in some cases, for the formation of amorphous co-precipitates by co-spray drying a drug with a crystallization inhibitor, e.g., to improve the bioavailability of hydrophobic drugs. In addition, as discussed herein, using independent injection of two solvent streams, drug co-precipitates can be prepared.

In conventional spray dryers, a single liquid stream is typically atomized by compressed air in a spray nozzle; the spray is then mixed with a heated gas stream in a drying chamber to evaporate the solvent and yield the dried product. However, this setup only allows processing of single solvent systems or mixtures of premixed solvents. To process multiple separate solvent streams as required for solvent/antisolvent precipitation or rapidly reacting solvent streams, the spray dryer can be outfitted with additional separate inlet channels. In this example, a microfluidic device with an array of two flow-focusing cross junctions is used, as is shown in FIG. 1, which shows a schematic diagram of a microfluidic device for forming nanoparticles from hydrophobic drugs by spray drying.

This device geometry allows for the separate injection of two solvent streams, and provides a third inlet for compressed air. For the formation of hydrophobic drug nanoparticles, the hydrophobic drug may be dissolved in an organic solvent, which is injected into the first inlet (“Solvent 1”), and the second fluid may be injected into the second inlet (“Solvent 2”). The two solvents may be injected in such a way as to form a jet at the first cross junction, which extends into the second cross junction where compressed air is injected (“Air”). The injection of compressed air may cause the fluids to form discrete droplets, e.g., a spray or a mist.

To process hydrophobic drugs, the PDMS device should resist fouling due to adsorption of drug crystals or other precipitants on the microchannel walls. This was achieved in this example by treating the intrinsically hydrophobic PDMS device with oxygen plasma, as the plasma renders the spray dryer channels more hydrophilic. Although the hydrophilicity of the plasma treated device decreases over time, the channel surface can be regenerated in the same manner, e.g., multiple times. To further improve the resistance against fouling, the surface contact was minimized between the drug-loaded solvent stream and the channel walls. This can be achieved by designing a device geometry with a high aspect ratio. The aspect ratio (height to width of the channel) was 10:1 in the upper half of the device and 4:1 at the spray nozzle. As high aspect channels are less pressure-resistant than square channels, the spray dryer channels may expand somewhat in some cases, as shown in FIG. 2.

To determine the impact of the channel deformation on the flow profile, a typical solvent/antisolvent system was processed in an inventive spray dryer and the device deformation within the device was compared at low and high pressure. These observations were supported by computational fluid dynamics (CFD) simulations using COMSOL 4.0a. A 3D simulation model was designed considering the structural mechanics of the PDMS channels, the fluid flow described by the Navier-Stokes equations and the diffusion of the solvent streams.

For the spray experiment at low pressure, a solvent (isopropyl alcohol, IPA), an antisolvent (water), and compressed air was injected into the first, second, and third inlets, respectively, at flow rates of 1 ml h⁻¹ for IPA and 10 ml h⁻¹ for water. The air pressure was set to 0.34 bar, as shown in FIG. 2A. For the high-pressure experiments, the flow rates of IPA and water were increased to 5 ml h⁻¹ and 50 ml h⁻¹, respectively, and the air pressure was set to 2.09 bar, as shown in FIG. 2B. At low pressures (0.34 bar), the PDMS device demonstrated minimal deformation and a two dimensional focused flow pattern was observed between the first and second cross junction. However, as the pressure was increased, the PDMS device responded to the internal stress and the channels expanded slightly. Due to the high aspect ratio, the strongest expansion of the microchannels was observed in horizontal direction lateral to the fluid flow; the channel walls adapted a quasi-circular shape.

This deformation influenced the flow profile inside the spray dryer, as shown in FIG. 2C. The impact of the deformation on the flow profile was studied using CFD simulations. As illustrated by the simulation of the device, the flow profile between the first and second cross junction adopted a three dimensional coaxial flow pattern. As shown in FIG. 2C, the initial rectangular microchannels expanded and adopted a quasi-circular shape. As illustrated by the simulation of the device, the flow profile between the first and second cross junction adopted a three dimensional coaxial flow pattern, therefore reducing the contact surface between the drug-loaded solvent stream and the channels walls. The scale bars denote 100 micrometers. Thus, the inner phase was surrounded by a protective sheath of the middle phase. This minimized the surface contact of the solvent in which the hydrophobic drug is dissolved with the channel walls and prevents fouling of the spray dryer.

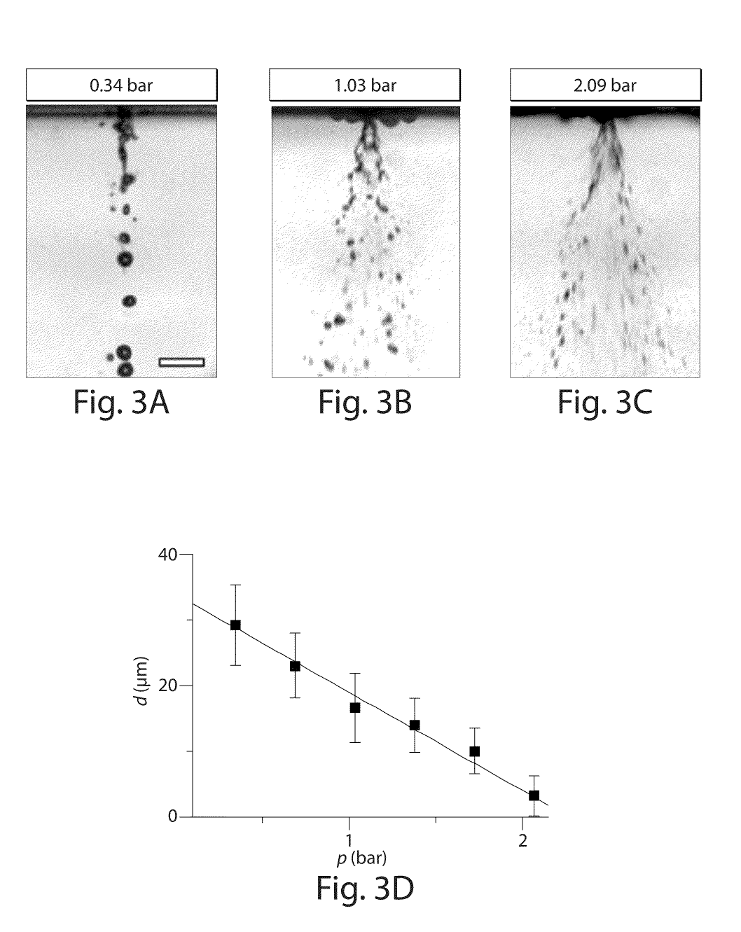

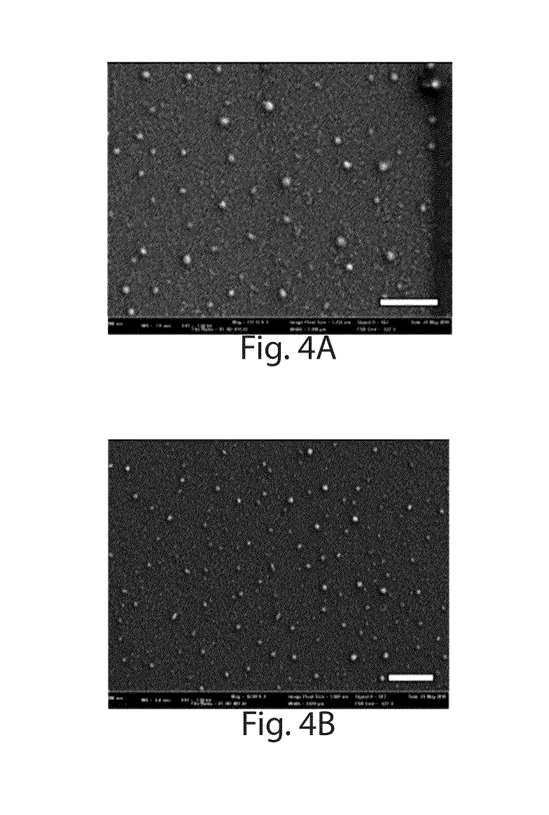

When forming a spray, the spray shape and drop size are important factors influencing drying, particle size and morphology of the processed drug. To determine drop size and spray shape, the spray formation in the spray dryer was visualized by recording movies with a high-speed camera. IPA was injected into the first and second inlet at a total flow rate of 50-55 ml h⁻¹. At low air pressure, a fluid jet is ejected from the nozzle which breaks into single droplets downstream, and the solvent stream was not dispersed into a spray; instead, a jet of liquid was ejected from the spray nozzle and broke into large droplets due to Rayleigh-Plateau instability, as shown in FIGS. 3A-3C. The scale bar for all panels denotes 100 micrometers. As the air pressure was increased beyond 0.5 bar, the formation of finely dispersed drops at the spray nozzle was observed, which adopted a round full cone spray pattern. This pattern was formed due to turbulences imparted to the liquid prior to the orifice in the short outlet channel.

To quantify the spray formation process, the drop size (diameter) d was measured as a function of the air pressure p, as shown in FIG. 3D. With increasing pressure, the mean size of the droplets decreases linearly. The line is a guide to the eye. The drop size decreased linearly with increasing pressure to approximately 4 micrometers in diameter at 2.1 bar, which is the maximum pressure this particular spray dryer could withstand without delamination of the plasma-bonded PDMS. However, in other embodiments, higher air pressures may be achieved, e.g., by increasing the spacing between the microchannels and, therefore, the pressure resistance of the PDMS device.

### EXAMPLE 2

This example illustrates the formation of hydrophobic drug nanoparticles using a microfluidic spray dryer according to one embodiment of the invention. In this example, danazol was used as a model drug. Danazol is an isoxazole derivative of testosterone and applied for the treatment of endometriosis and hereditary angioedema. It has the following structure:

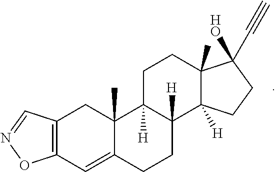

One method for processing hydrophobic drugs is liquid antisolvent precipitation (“LASP”), where the drug, dissolved in an alcohol, is precipitated by mixing the drug solution with water as the antisolvent. In this example, danazol was dissolved in isopropyl alcohol and then injected together with water into a first cross junction. As the microfluidic device was operated in a laminar flow regime, only diffusion-based mixing of the solvent streams was observed at their interface, which did not lead to any precipitation of the drug.

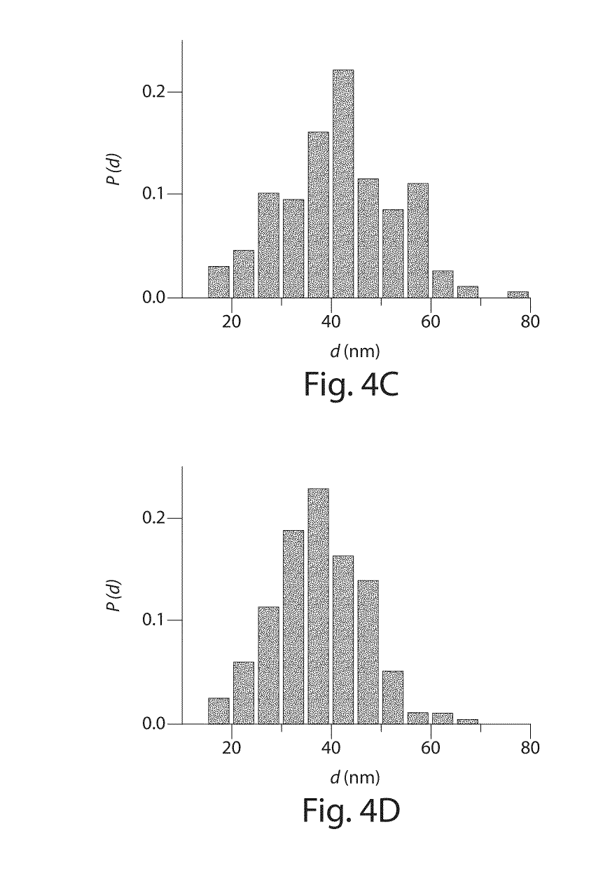

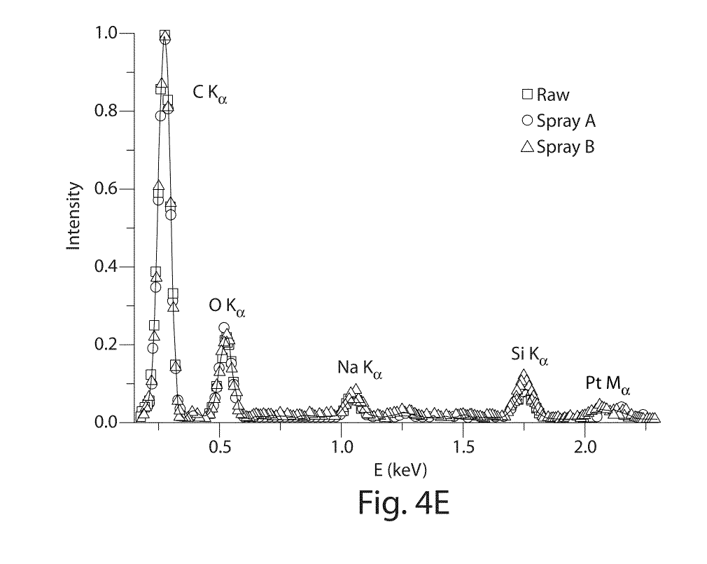

To evaluate the effect of microfluidic processing on particle size and morphology of the hydrophobic drug, no stabilizer or surfactant was added to influence the particle growth. The flow rates were initially set to 5 ml h⁻¹ for danazol, and 50 ml h⁻¹ for water, which corresponded to a volumetric ratio of 1:10 and has been shown to yield danazol microparticles in conventional LASP processes The spray in this example was completely suspended in air, thus ensuring that the product dried upon collection. Morphology and particle size of the processed drug was examined by scanning electron microscopy (“SEM”) analysis. While unprocessed (raw) danazol is composed of particles with irregular shapes ranging from approximately 2 micrometers to 100 micrometers, the particle size was decreased significantly in this example by processing the drug using the microfluidic spray dryer. As shown in FIGS. 4A and 4C, danazol nanoparticles were obtained having a narrow particle size distribution (“PSD”) from 20 nm to 60 nm, smaller than previously reported. The scale bar denotes 300 nm.

The formation of drug nanoparticles using LASP was driven by mixing of the drug solution with the antisolvent. The degree of supersaturation of the drug solution appeared to govern nucleation and growth of the drug nanoparticles. However, sufficient mixing only occurred in the short outlet channel prior to the orifice of the spray nozzle in our microfluidic device. As high flow rates were used to form a stable spray, the delay time of the fluids in the outlet channel should be too short to enable growth of nuclei by mixing. Thus, to further study the formation process, the antisolvent (water) was replaced by the solvent (IPA) in certain experiments, where a solution of danazol in IPA and pure IPA was respectively injected into the first and second inlet of the microfluidic device.

As can be seen in FIGS. 4B and 4D, danazol nanoparticles of identical size and morphology were obtained. Scale bar denotes 300 nm. The formation of danazol nanoparticles of identical size and morphology in the absence of the antisolvent suggested that particle formation thus was primarily driven by the evaporation of the spray, and not by the formation of nuclei due to supersaturation. The energy dispersive X-ray (“EDX”) analysis (FIG. 4E) of unprocessed (“raw”) and processed danazol (“spray”) also showed that the chemical composition of the drug remained unchanged during the spray drying process using either solvent system.

### EXAMPLE 3

Another important parameter of the spray drying process is the collection distance of the final particles from the nozzle through the drying region. While it is known that the morphology and size of hydrophobic drugs is dependent on the initial concentration of reactants, the choice of additives, the ratio of solvent and antisolvent, etc., it was found, by performing spatial sampling of the spray as discussed herein, that there was also a significant dependence on the collection distance.

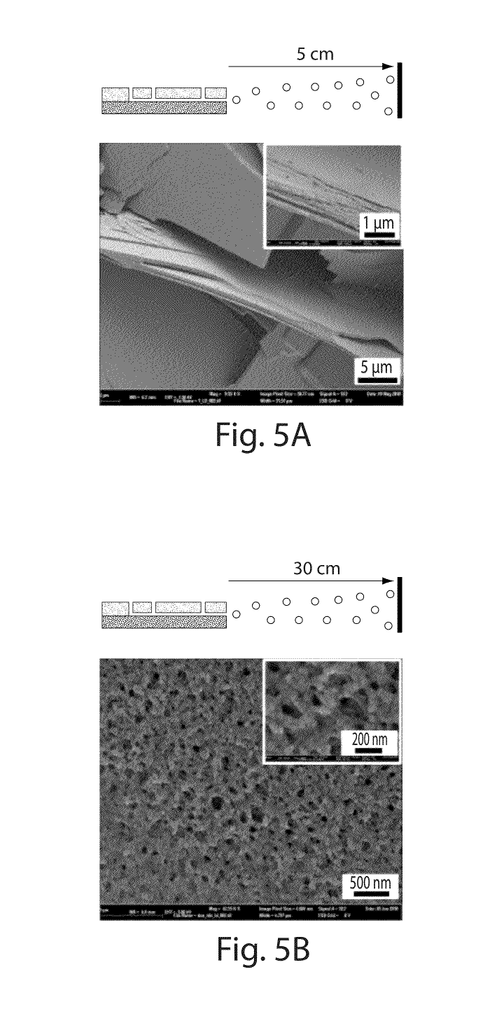

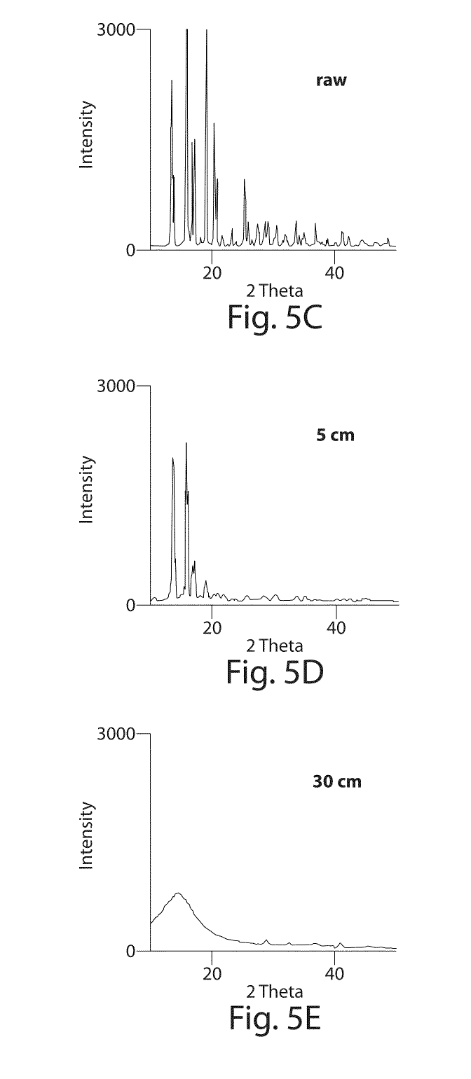

To illustrate this, in this example, danazol together with IPA was injected as before, but this time, the spray was collected in steps of 5 cm from the spray nozzle. SEM analysis was performed, revealing two distinct product morphologies. At a collection distance of 5 cm, a layer-by-layer assembly of danazol was observed; the thickness of each layer is 60 nm to 80 nm, as shown in FIG. 5A. These values were in good approximation with the size of single danazol nanoparticles, as shown in FIG. 3.

However, as the time of flight was too short to allow for complete evaporation of the spray, the remaining solvent increases the mobility of particles already formed, allowing them to fuse and reach an energetically more favorable state. Thus, the collection distance was increased to 30 cm; as the spray was completely evaporated, single nanoparticles were formed, as shown in FIG. 5B, showing nanoparticles, approximately 20 nm to 60 nm in diameter, assembled in a dense network.

X-ray powder diffraction analysis (“XRPD”) was employed to determine the effect of spatial sampling on the crystallinity of danazol. Characteristic peaks at 20 (2 theta) of 15.8, 17.1 and 19.0 in the XRD pattern of unprocessed danazol were used as reference. In processed danazol, the intensity of the characteristic peaks decreased as the collection distance of the spray is increased. This suggested that the initial crystallinity of the drug was not recovered, as shown in FIGS. 5C-5E. These figures show nanoparticles, approximately 20 nm to 60 nm in diameter, assembled in a dense network. The formation of amorphous danazol is of importance, as the difference in physicochemical properties of the amorphous form may significantly increase the bioavailability of danazol in some embodiments.

### EXAMPLE 4

Another way to fabricate amorphous hydrophobic drugs is to co-spray dry the drug and a crystallization inhibitor. This example demonstrates this using a microfluidic spray dryer.

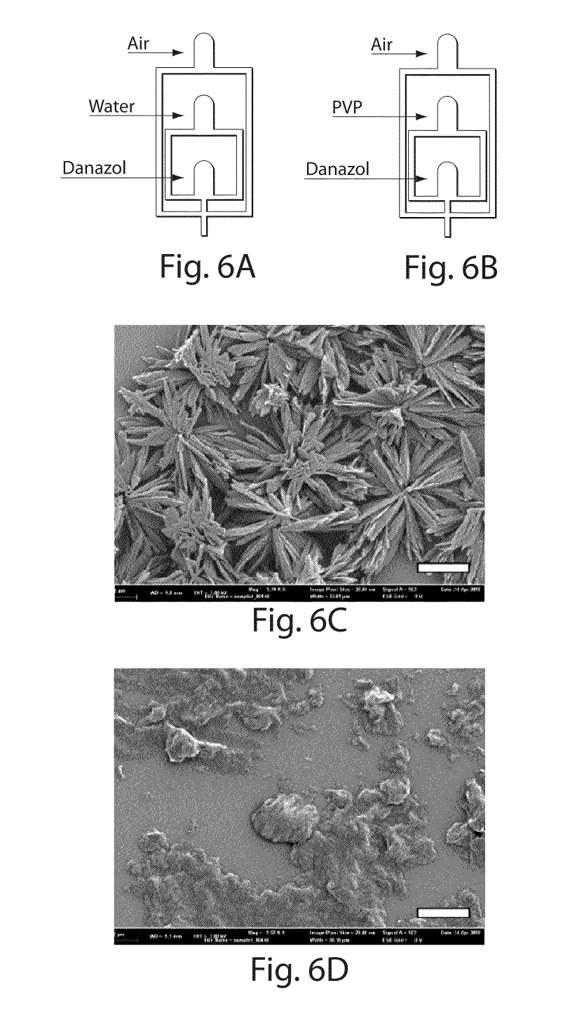

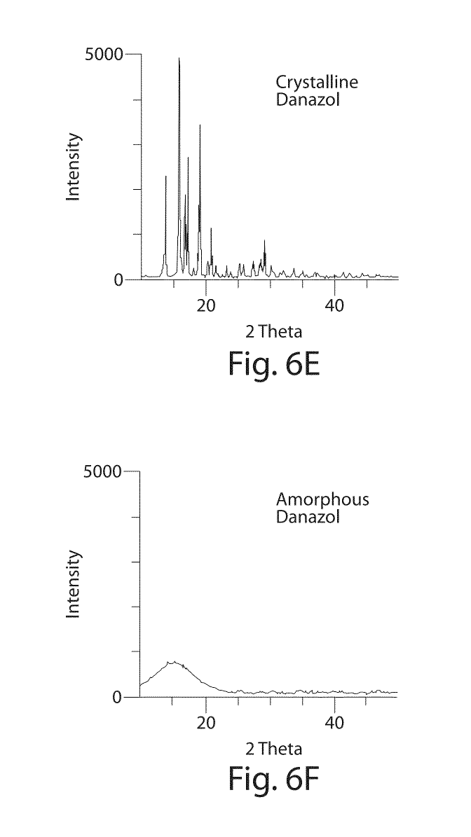

In one experiment, dry danazol in IPA was co-sprayed together with water and the spray collected at a low distance, as is shown in FIG. 6A. In particular, the danazol in IPA was mixed with water inside the microfluidic device, and the spray was collected at a distance of 5 cm from the nozzle, allowing danazol to grow into crystalline aggregates, as indicated by the XRPD pattern. The spray did not appear to be completely evaporated due to the short time of flight. This allowed danazol to grow into star-shape crystalline aggregates, as shown in FIGS. 6C and 6E. However, in other experiments, by using a crystallization inhibitor, amorphous danazol may be formed. Poly(vinylpyrrolidone) (PVP) is well known to inhibit crystal growth in pharmaceutical formulations, and was used in these experiments. Danazol in IPA was processed together with a 1.5 wt % solution of PVP in water at equal flow rates of 25 ml h⁻¹, as shown in FIG. 6B. Again, the spray was collected at a short distance. However, as the spray was dried, danazol precipitated from the spray in a PVP matrix without crystallization as shown in FIG. 6D. Thus, no characteristic peaks were observed in the XRPD pattern, as is shown in FIG. 6F (compare to FIG. 6E). Scale bars in FIGS. 6C and 6D denote 5 micrometers.

### EXAMPLE 5

This example compares experiments performed using a spray dryer as discussed herein with the same formulations in a conventional laboratory spray dryer. The results are compared using XRPD (X-ray powder diffraction analysis) and SEM.

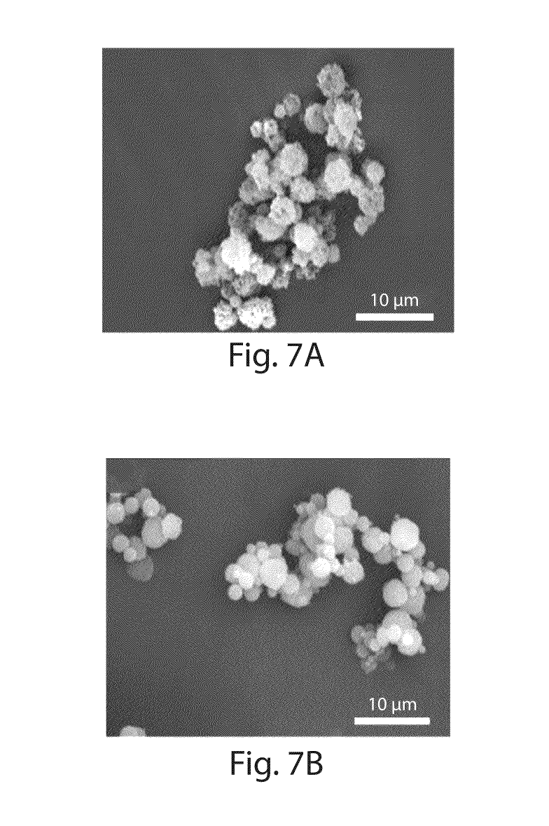

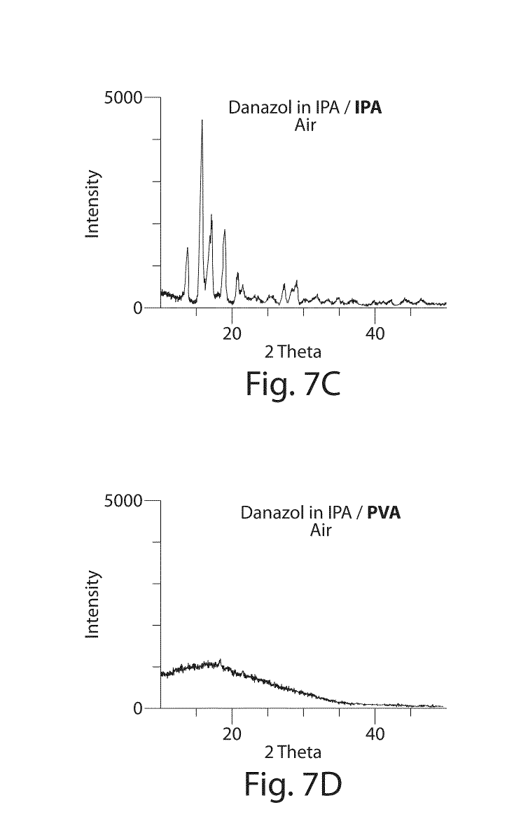

In these experiments, a Mini Spray Dryer B 191 (Büchi, Germany) with a spray rate of 10 mg min⁻¹ was used, and a solution of danazol in IPA as well as a solution of danazol in IPA together with PVP was tested. In the former case, particles ranging from approximately 1 micrometers to 5 micrometers were produced (FIG. 7A), and, these were substantially larger than the danazol particles formed using the microfluidic spray dryers discussed above. In addition, the crystallinity of danazol was retained, as shown in FIG. 7C. Similar results were observed for the formation of co-precipitates of danazol and PVP, as shown in FIGS. 7B and 7D. Although the initial crystallinity of danazol was suppressed by PVP, the particles were again two orders of magnitude larger than in comparable experiments using the microfluidic devices discussed above.

### EXAMPLE 6

This example illustrates various techniques used in the previous examples. The PDMS microfluidic devices were fabricated using soft lithography. All channels had a fixed height of 100 micrometers. The PDMS replica was bonded to a flat sheet of cured PDMS using oxygen plasma treatment. The plasma treatment rendered the microchannels temporarily hydrophilic. To retain the hydrophilic surface modification, suitable for handling hydrophobic drugs, the device was flushed with deionized water. The nozzle of the spray dryer was prepared by slicing the outlet channel of the stamped device with a razor blade. To achieve reproducible accuracy when slicing, a guide to the eye in the initial AutoCAD design was used in the spray dryer.

Spray drying experiments. PVP (weight-averaged molecular weight, MW 10,000 g mol⁻¹) and all other chemicals were obtained from Sigma-Aldrich Co. unless noted otherwise. Danazol (99.9%) was obtained from Selectchemie AG. Water with a resistivity of 16.8 MS2 cm⁻¹ (megohm/cm) is prepared using a Millipore Milli-Q system. All solutions were filtered through a 0.2 micrometer PTFE filter (Millipore). To demonstrate long term stability of the process of forming danazol nanoparticles in a microfluidic spray dryer, each experiment was performed over a time period of 2 h. A saturated solution of danazol in IPA was injected into the first inlet and water or IPA was injected into the second inlet at 5 ml h⁻¹ and 50 ml h⁻¹, respectively. For the formation of co-precipitates, PVP in water (1.5% w/w) was injected at 50 ml h⁻¹ into the second inlet. To form the spray, air was injected into the third inlet at 2.1 bar. The spray was ejected into air and dried at room temperature; the yield ranged from about 70% to about 95%, depending on the experiment and the experimental conditions. The spray was imaged using a Phantom v9.1 camera (Vision Research) at 64,000 fps. The droplet size was obtained by measuring the size of at least 200 drops from high-speed camera images.

Processed danazol was collected at distances between 5 cm and 30 cm from the spray nozzle. For SEM analysis, the spray was collected on glass slides and coated with Pd/Pt. An Ultra55 Field Emission SEM (Zeiss) coupled with an EDX detector was used. The size distribution of the nanoparticles was determined by image analysis of SEM photographs using ImageJ. For XRPD analysis, samples were collected in an aluminum box over which the spray dryer was mounted. XPRD analysis was performed using a Scintag XDS2000 powder diffractometer (Scintag, Cupertino, California, USA) with Cu Ka (K-alpha) radiation at 40 kV and 30 mA. The XRD patterns were taken at room temperature in the range of 10°≦2θ≦50° (2 theta) with a scan rate of 1° min⁻¹ and a step size of 0.02°.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-83. (canceled)

84. A method of spray drying, the method comprising:
flowing a first liquid through a first microfluidic channel having an opening as a nozzle, wherein the first liquid comprises a first species dissolved in the first liquid;
flowing a second liquid through a second microfluidic channel intersecting the first microfluidic channel at an intersection upstream of the nozzle;
exposing the first liquid to the second liquid within the first microfluidic channel, wherein the first liquid is surrounded by the second liquid downstream of the intersection such that the first liquid does not contact a wall of the first microfluidic channel proximate to the nozzle, wherein the first species precipitates upon exposure of the first liquid to the second liquid; and
spraying the first liquid and the second liquid into a drying region external of the first and second microfluidic channels.

85. The method of claim 84, wherein the first liquid and the second liquid are immiscible.

86. The method of claim 84, further comprising flowing the second liquid through a third microfluidic channel intersecting the first microfluidic channel at the intersection upstream of the nozzle.

87. The method of claim 84, further comprising flowing a third fluid through a fourth microfluidic channel intersecting the first microfluidic channel at a second intersection downstream of the intersection of the first microfluidic channel and the second microfluidic channel.

88. The method of claim 87, wherein the third fluid comprises air.

89. The method of claim 87, further comprising flowing the third fluid through a fifth microfluidic channel intersecting the first microfluidic channel at the second intersection.

90. The method of claim 84, wherein the first microfluidic channel has an average cross-sectional dimension of less than about 1 mm.

91. The method of claim 84, wherein the opening has a cross-sectional aspect ratio of at least about 3:1.

92. The method of claim 84, wherein the first microfluidic channel has a cross-sectional aspect ratio of at least about 5:1.

93. The method of claim 84, wherein at least a portion of the first microfluidic channel is coated with a hydrophilic coating.

94. The method of claim 84, wherein at least a portion of the first microfluidic channel is hydrophilic.

95. The method of claim 84, wherein the first species is a pharmaceutical agent.

96. The method of claim 84, wherein the first liquid and/or the second liquid comprise a crystallization inhibitor of the first species.

97. The method of claim 84, further comprising collecting particles in the drying region, the particles comprising the first species.

98. The method of claim 97, wherein at least a portion of the first species within the particles is not crystalline.

99. The method of claim 84, wherein the second liquid comprises a second species dissolved in the second liquid.

100. The method of claim 84, wherein the first liquid and/or the second liquid flow laminarly through the first microfluidic channel.

101. The method of claim 84, wherein the first liquid is exposed to the second liquid within the first microfluidic channel for a period of time of no more than 15 seconds.

102. The method of claim 84, further comprising exposing the first liquid and/or the second liquid to an electric field to cause the first liquid and the second liquid to form droplets within the drying region.

103. The method of claim 84, further comprising heating the drying region to a temperature of at least about 40° C.
